(19) United States
(12) Patent Application Publication
(10) Pub. No.: US 2010/0298834 A1
(43) Pub. Date: Nov. 25, 2010

(54) DEVICE AND APPARATUS FOR PERFORMING AN ENDOPROSTHESIS IMPLANTATION

(76) Inventor: Bernhardt HILDEBRANDT, Bad Wildbad (DE)

Correspondence Address: CANTOR COLBURN LLP, 20 Church Street, 22nd Floor, Hartford, CT, 06103, US

(21) Appl. No.: 12/785,656

(22) Filed: May 24, 2010

Related U.S. Application Data
(63) Continuation-in-part of application No. PCT/DE2008/001965, filed on Nov. 26, 2008.

(30) Foreign Application Priority Data
Nov. 27, 2007 (DE) 10 2007 057 008.4
Dec. 22, 2007 (DE) 10 2007 062 640.3
Jul. 7, 2008 (DE) 10 2008 031 957.0
Oct. 28, 2008 (DE) 10 2008 053 566.4

(51) Int. Cl. A61B 17/16; A61B 17/88; A61B 17/90

(52) U.S. Cl. 606/80; 606/96; 606/99; 606/102; 606/86 R

(57) ABSTRACT

Through the devices and apparatuses that are provided for performance of an implantation of endoprostheses, the previously unavoidable soft tissue damage and bone loss are largely reduced by carrying out the implantation via a narrow joint gap and a hole through the bone and through the rotational center point of the joint head. A fixation device and an extension arm of an extension device guarantee a bore true to angle, the system bore, through the proximal femur and femur head or humerus and humerus head. These must be extended with an extension device only by 2.0 to 2.7 cm in the bent position from the joint socket and are pivoted into the extension position. A narrow soft tissue access is therefore sufficient in order to introduce milling cutters and prostheses into the joint by means of a gripping tool. Through the system bore the drive shafts of the milling cutters as well as the press-in rods of the socket shell and the head prosthesis are guided true to angle and the latter pressed in by the press-in method and fixed by a fixation rod and counter plate. The devices are suitable for endoprosthesis implantations of the shoulder and hip.

### CROSS REFERENCE TO RELATED APPLICATIONS

The present application claims the priority of German patent applications 10 2007 057 008.4, filed on Nov. 27, 2007, 10 2007 062 640.3 filed on Dec. 22, 2007, 10 2008 031 957.0 filed on Jul. 7, 2008 and 10 2008 053 566.4 filed on Oct. 28, 2008 the entire content of all of which is hereby incorporated by reference. Furthermore, the present application is a continuation in part application of PCT patent application serial no. PCT/DE 2008/001965 filed on Nov. 26, 2008, the disclosure content of which is also incorporated herein by reference in its entirety.

### FIELD OF THE INVENTION

The invention concerns an apparatus for performing hip or shoulder joint endoprosthesis implantation. The invention also concerns devices usable with this apparatus, like a milling cutter, a prosthesis shell holder, a prosthesis for a joint head, an inspection tool to inspect reaming, a pivot-gripping tool to pivot in the milling cutter and prostheses into a joint gap created by the apparatus, a measurement probe to establish milling cutter guiding relative to the reaming depth on the joint head and an apparatus for extension of the joint capsule.

### PRIOR ART

Endoprostheses have the task of replacing worn joints. Present endoprostheses have very limited lifetime and loadability. In the shaft and short shaft prostheses and pressure disk prostheses this is primarily due to the non-physiological force introduction of the prosthesis into the bone. In surface replacement prostheses it is due to the fact that serious soft tissue damage is connected with the operation. The femur head must be levered outward for treatment. This is only possible after prior removal of the joint capsule in separation or tearing of the short hip musculature. Precision of femur head milling also could not be optimized thus far so that the cemented anchoring (McMinn et al.) achieves the best result, in which case, however, only incomplete bone connection can be achieved and cement aging, for instance, limits the lifetime (see, for example, Swedish Hip Register).

For the shoulder joint there are also no apparatuses and prostheses at present that permit implantation without adverse injuries: for example, the musculus subcapularis and part of the joint capsule as well as the rotator cuff must be detached. This requires long rehabilitation in the shoulder with long inactivity (a total of about 6 months) and, in addition to other complications, entails the hazard of tearing of the tendons of the shoulder musculature separated and re-attached during the operation.

### SUMMARY OF THE INVENTION

The present invention sets itself the task of creating devices for an endoprosthesis implantation method which avoids all the aforementioned drawbacks in which treatment of the joint surfaces and implantation of the prostheses occur through a narrow joint gap and the milling shafts and press-in/-on rods are driven and precisely guided by a tool guide and system bore.

This task is solved by the apparatuses for performing a hip or shoulder joint endoprosthesis implantation as well as by the devices usable with this apparatus, like a milling cutter, prosthesis shell holder, a prosthesis for a joint head, an inspection tool to inspect reaming, a pivot-gripping tool to pivot the milling cutter and prostheses into a joint gap created by the apparatus, a measurement probe to establish milling cutter guiding relative to the milling depth on the joint head and an apparatus to extend the joint capsule.

Tools and apparatuses that correspond to those described in the patent claims are not known and a search gave no indications of similar patent applications.

Product names and manufacturers of endoprostheses for the shoulder and hip:

BHR—distributor of the Smith & Nephew Co. (manufacturer Finsbury Co.)
ADEPT—manufacturer and distributor of the Finsbury Orthopaedics Co.
Durom—manufacturer and distributor of the Zimmer Co.
ASR—manufacturer and distributor of the DePuy Co.
Cormet 2000—manufacturer and distributor of the Corin Co.
Conserve Plus—manufacturer and distributor of the Wright Medical Co.
Bionik—manufacturer and distributor of the ESKA Co.
Icon—manufacturer and distributor of the International Orthopaedics Co.
Accis—manufacturer and distributor of the Implantcast Co.

Patent searches relevant to the hip joint:

### DE 101 30 366 A1, US 2003/014123 A1, DE 30 06 178 C2, EP 1 260 200 A

relevant to the shoulder joint (+hip joint): EP 1 566 154 A

The Medacta Co. has developed an extension apparatus for implantation of hip joint endoprostheses (mobile leg positioner, U.S. Pat. No. 7,316,040 A and EP 1 604 629 A). The AMIS access (anterior-minimal-invasive surgery in total hip replacement) is therefore made possible on the hip. It does permit retention of the joint capsule and only limited soft tissue damage, but only for implantation of shaft prostheses, since the access to the acetabulum only becomes possible after removal of the femur head. The extension apparatus employed therein sits on the foot of the extended leg and has neither the system center adjustment apparatuses according to the invention, nor a system axis pivot device, nor a control device, nor an extremity holder adjustable relative to the extension arm. It does have an ischium-pubic bone support, but does not have the other components of the hip fixation according to the invention that fix the pelvis.

The devices and prostheses determined in the aforementioned patent searches, like all known implantation methods, require the usual accesses with the corresponding soft tissue damage, which occurs owing to the fact that the joint head must be levered from the cavity or removed in order to gain access to the cavity and to treat the joint head. They do not have the advantages of the method made possible by the apparatuses according to the invention.

Further advantages are apparent from the dependent claims and the following description.

The invention is explained in detail below by means of practical examples shown in the appended figures.

The principle of the implantation method made possible by the devices and apparatuses according to the invention consists of the fact that treatment of the joint surfaces and implantation of the prostheses 21h, 22a occurs through a narrow joint gap, which is produced by an extension device 17. The milling cutter shafts 19b, 19m, 20d, 20r and press-in/-on rods 21e, 22m of the prostheses are aligned and guided by system axis pivot devices 17b-17l, 7n with reference to spatial arrangement and angle relative to joint 9b, 11a by a control device 13 on a system axis 13y. System center adjustment devices 2-2c, 17a-17c, 17l, 7n, 14b-14d permit adjustment of the system center 13w and the system axis 13x to the joint center point 8b, 11ab, which is a prerequisite for guiding of the tools, for the extension and pivots to introduce the milling cutter 19, 20 and prostheses 21h, 22a into the joint gap. The system axis 13x of the extension device 17 is defined by the fact that it runs parallel to the extension guide 17l, 17m, 17ma, 13dj, 13da-13do, corresponds to the adjusted axis of the tool guide 13a of the control device 13 and runs through the system center 13w. The system center 13w is defined by the fact that it is the intersection point of at least one, preferably all axes of a system axis pivot device 17b-17l, 7n with the system axis 13w.

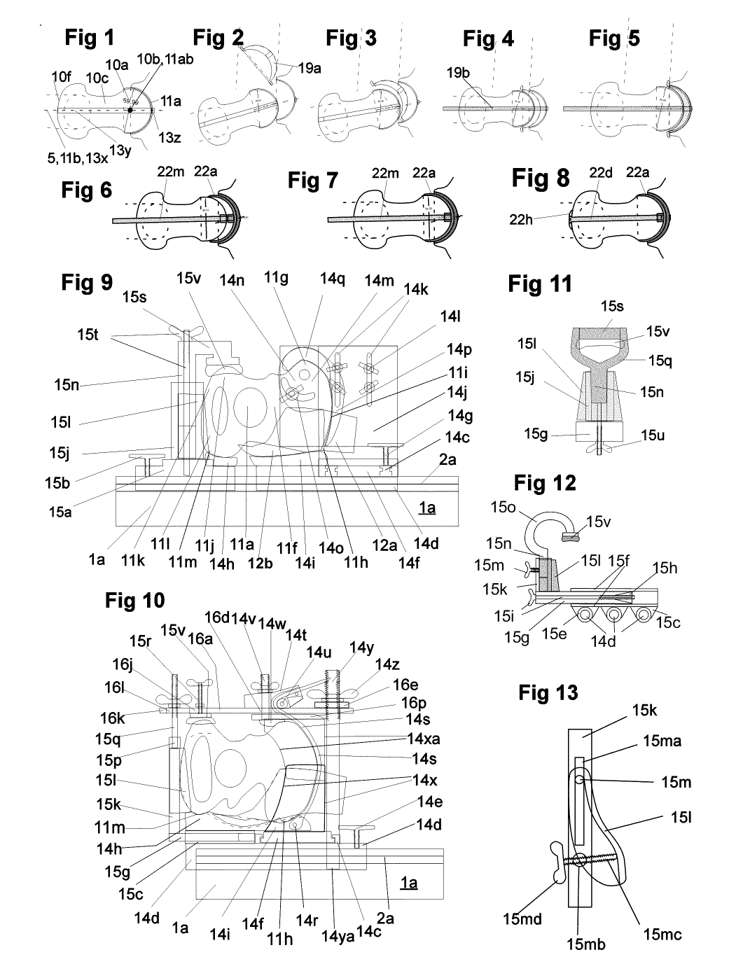

FIG. 1 to FIG. 8 show on the example of a hip prosthesis implantation with anterior access the concept of the implantation process.

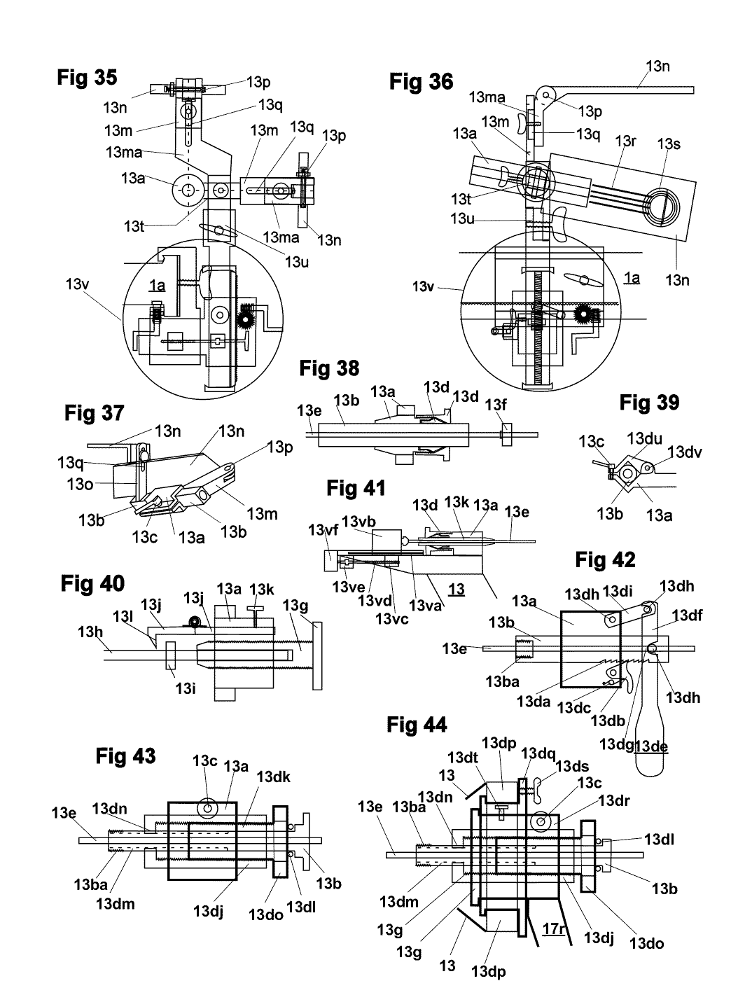

A control device 13 according to FIGS. 35-44 with a tool guide 13a on the system axis ensures exact positioning of the system bore 13y through the humerus 8c or femur 10j into the joint.

FIG. 1 shows a view of the status after making the system bore 13y through the femur shaft 10f, the femur neck 10c and the femur head 10a, which has a continuation 13z in the acetabulum 11a (this extension 13z serves as a guide for milling cutter shafts 19b, 19m, 20d, 20r). The extremity extension brace 17p, 17w-17z, 17za-17zr mounted on the extension device 17 according to the invention or on a control device 13 combined with it holds the extremity so that the joint head 8a, 10a can be extended from the socket 9b, 11a. To avoid injury or tearing of the capsule-ligament apparatus the extension must be performed in the bent position of the extremity, since the joint capsule is then relieved and permits the largest possible joint gap. This requires positioning of the extremity extension brace 17p, 17w-17z, 17za-17zr on the humerus or femur. A joint gap wide enough (about 2.2-2.7 cm) to accommodate the milling cutters 19, 20 and prosthesis shell holder 21 according to the invention adapted to this joint gap can thus be created. To introduce them into the joint gap the extended joint head is pivoted to one side of the joint by the system axis pivot devices 17b-17l, 7n so that the joint gap is opened wider on the other side.

FIGS. 2-3 show this extension of the joint head through an extension device 17 according to FIGS. 24-34 and pivoting to create a gaping, narrow joint gap. No space remains in the joint gap to drive the milling cutter shafts 19b, 19m, 20d, 20r and press-in/-on rods 21e, 22m for the endoprostheses. The milling cutter shafts 19b, 19m, 20d, 20r are inserted according to the invention through a system bore 13y in the milling cutter bodies 19a, 20a and prosthesis shell holder 21a and prosthesis shells 21h, 22a. These are brought into their working position by back-pivoting (FIG. 4). Milling cutter shafts 19b, 19m, 20d, 20r and press-in/-on rods 21e, 22m are introduced through the system bore 13y and the control device 13 according to FIGS. 35-44 by the tool guide 13a of the control device 13 and aligned in so doing exactly to the planned anteversion and inclination angle 5 (FIGS. 4-7). Much more precise milling (FIG. 5) and pressing in and pressing on of the prostheses are achieved than in the known methods.

FIG. 6 shows the beginning of the press-on process of a surface replacement prosthesis 22a of the femur head 10a (with the socket prosthesis already in position) and FIG. 8 shows a surface replacement prosthesis 22a of the femur head 10a anchored by a fixation rod 22d with counter plate 22h, whose anchoring is more exact and more stable than in the known methods.

For the aforementioned processes a precise position and direction relation between joint socket 9b, 11a, the joint head 8a, 10a being extended, extension device 17 and control device 13 is necessary. This requires:
Exact fixation of the pelvis 11 or shoulder blade 9a (and with it the corresponding joint center point 8b, 11ab)—also relative to the extension tension and relative to the press-in pressure during socket implantation. This occurs through the fixation device for the pelvis 14-16 or shoulder 7-7w.
Exact localization of the joint center point 8b, 11ab. This occurs through x-ray templates 13n, 17ci, 17u and a C-arc x-ray device.
Adjustment of the system center 13w to the determined joint center point. This occurs through the system center adjustment device 2-2d, 17b, 17c, 17l, 7n on which the extension device 17 is mounted relative to the fixation device 7-7w, 14-16.
Adjustment of the direction of the system axis 13x and the extension guide 17l, 17m, 17ma, 13dj, 13da-13do to the planned inclination and anteversion angle 5 (which generally agrees with the angle of the joint axis 9c, 11b). This occurs through the system axis pivot devices 17b-17l, 7n of the extension device 17 (the same things that also carry out pivoting to produce the joint gap).
Adjustment of the axis of the tool guide 13a of the control device 13 to the system axis 13x is already present with the combination of extension arm 17 and control device 13, otherwise this occurs through adjustment devices 13v, 13u, 13t on control device 13.
Adjustment of the axis of the joint head or femur neck to the system axis 13x (for example, to make the system bore 13y) occurs through the pivot and/or adjustment devices 17q, 17ra-17rg, 17ri, 17rj, 17rm, 18, 18a-18h of the extremity brace 17r (which is mounted on the extension arm 17o).

In order to precisely fix the hip or shoulder relative to the extension tension and the press-in pressure relative to a control device 13 that guides the tools and the extension device 17-18, a fixation device 14-16 for the pelvis or shoulder 7, 7a-7w is required. The fixation device 14-16, 7, 7a-7w is constructed on a base 14d, 7, which is connected to the extension device 17-18 adjustable in the three spatial directions via the system center adjustment devices 2, 2a-2d, 17b, 17c, 17l, 7n. One of the system center-adjustment devices 2, 2a-2d, 17b, 17c, 17l, 7n or the base 14d, 7 or support plate 7o of the holding devices or extension device 17 has connections 1a to the operating table 1. As a substitute, the supports of the holding devices 14-16, 7a-7w can assume adjustment functions, for example, in that a rear pelvic brace 14i is adjustable in height and/or the lateral pelvic supports 14x, 14xa, 14zl, their holders 14j, their foot plate 14f are adjustable transversely and/or longitudinally or the holding devices 7d-7m, 7p-7u of the shoulder fixation 7b-7t are correspondingly adjustable and/or the snap-in device 7n of the support plate 7o of the shoulder fixation 7 has a pivot and/or longitudinal guide.

To set the planned inclination and anteversion angle 5 and for pivoting into the extension position (to produce a joint gap gaping on one side) the extension device 17 has at least two system axis pivot devices 17b-17l, 7n. The system center adjustment devices 2-2d, 17b, 17c, 17l, 7n could be integrated in the system axis pivot devices 17b-17l, 7n, for example, in that an axial shaft is used both for pivoting and for axial displacement. In FIGS. 23, 24, 25, 27, for example, a hollow cylindrical pivot guide 17b is involved in which the mounted hollow cylinder 17c is axially adjustable in height. The base 17a of the extension device in this variant is mounted, for example, via two longitudinal guides at right angles to each other in the operating table plane relative to the base 14d, 7 of the fixation device 15-16, 7 (similar to a cross table).

The task of the system center adjustment devices 2-2d, 17b, 17c, 17l, 7n is to adjust the system center 13w to the joint center point 8b, 10b. Adjustment can be accelerated by the fact that a holder 17n with a snap-in device 17na for the corresponding holder of a C-arc x-ray device is mounted on a system center adjustment device 2-2d, 17b, 17c, 17l, 7n on the operating table 1, on a connection 1a to the operating table 1 or on the extension device 17 so that after fastening of the x-ray device to this holder 17n a fixed position reference of the central x-ray beam to the system center 13w or joint center point 8b, 10b exists.

The control device 13 with the tool guide 13a serves for precise positioning of the system bore 13y and for application and guiding of the milling cutter shafts 19b, 19m, 20d, 20r and press-in/-on rods 21e, 22m, whose precise angular control is guaranteed by adjusting or aligning the tool guide 13a so that its axis coincides with the system axis 13x, i.e., passes through the joint center point 8b, 10b and coincides with the desired inclination and anteversion angle 5. The control device 13 is preferably connected to the extension arm 17o or forms a unit with it, in which case it can assume its functions. The axis of the tool guide 13a can be set fixed on the system axis on this account. The bushing 13b in the tool guide 13a in this case preferably has a receptacle 17z (for example, inside or outside thread, bayonet closure), with which the bushing 13b can be connected to the extremity extension brace 17p, 17w-17z, 17za-17zr, preferably in the form of an extension plate 17w-17z, 17za-17zr, i.e., the extension plate 17w-17z, 17za-17zr preferably has a central continuation of a corresponding receptacle structure 17z for the bushing 13b of the tool guide 13a or consists of one part with it. At least one longitudinal guide with the same axial direction as the tool guide can be mounted on the control device 13 or tool guide 13a or on a bushing 13b in the tool guide 13a, which serves, for example, as a slide 13va for longitudinal adjustment on the drive machines 13vb for drill 13e and/or milling cutter shafts 19b, 19m, 20d, 20r and/or press-in/-on rods 21e, 22m, in which the slide can be provided with a drive 13vf of the linear guide 13vc-13ve.

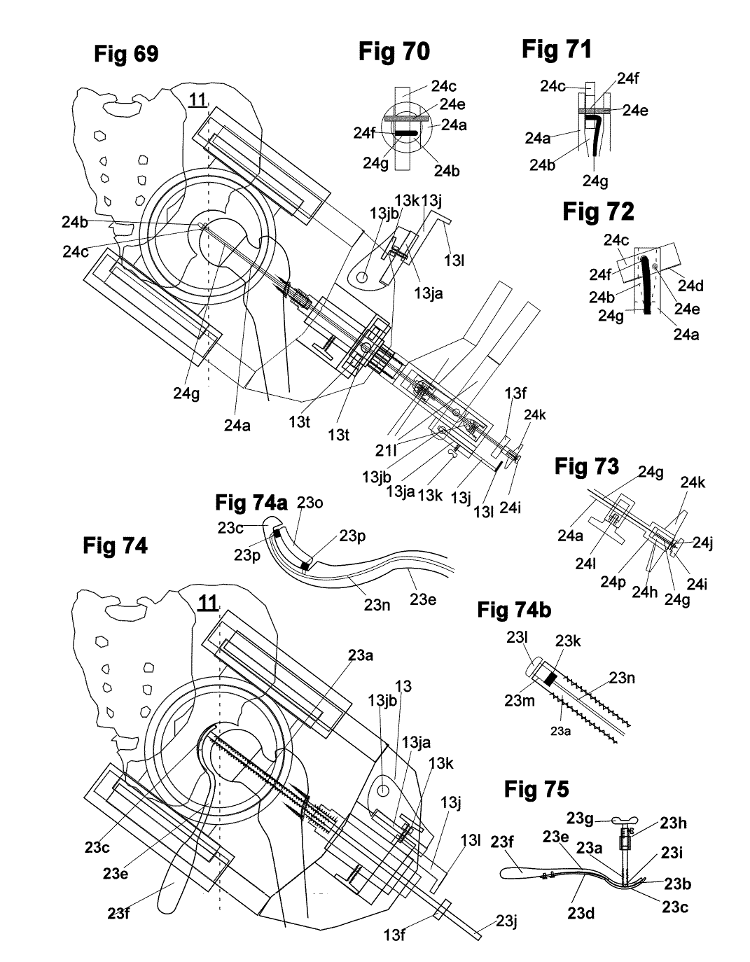

The tool guide 13a can be mounted fixed on the control device 13 or can be mounted with one or two pivot bearings 13t (for example, in a cardan suspension, see FIG. 69) and preferably can be fastened or snapped into the control device 13. The tool guide 13a can assume the function of the extension guide 17l, 17m, 17ma, 13da-13do, in which a bushing 13dj or the bushing 13b of the tool guide 13a mounted to move axially in it cannot only be displaced axially but also moved axially by a lever device 13da-13di, a screw guide 13dj, 13dk or other longitudinal guide. The tool guide 13a can also integrate a bearing 13dq, 13ds, 13t of the extremity extension brace 17r.

In order to facilitate covering with sterile cloths 13ub, the control device 13 can also have one or more matching insertion or snap-in connections 13u, 17oa to the extension arm 17o, through which it is fastened to it. For the same purpose the control device can also have one or more matching insertion or snap-in connections 17m to the extremity brace 17r. A control device substitute can then be provided for adjustment with a C-arc x-ray device, which is snapped in similar fashion on the extension arm 17 and secures the extremity brace 17r in a snap-in connection and is removed again after covering with sterile cloths and mounting of the sterile control device 17 on the extension arm 17o and snapping in of the extremity holder 17r on the sterile control device 17. In addition, a pivot bearing 13ta can be favorable for pivoting the upper part of the control device 13 with the tool guide 13a in order to facilitate access to the extremity. As an alternative the control device 13 can be mounted with the adjustment and pivot devices 13v on the operating table 1, on a connection 1a to the operating table 1, on one of the system center adjustment devices 2-2d, 17b, 17c, 17l, 7n or system axis pivot devices 17b-17l, 7n on the fixation device 14-16, 7 or the extension device 17.

System center adjustment devices 2, 2a-2c, 17b-17c, 17l, 7n, system axis pivot devices 17b-17l, 7n, extension devices 17l, 17m, 17ma, 13da-13do and pivot devices of the extremity holder 17q, 17ra-17rm, 18, 18a-18c, 18h as well as the longitudinal guide 13va-13vf of the control device 13 preferably have longitudinal guides or pivot guides (for example, screw guides or worm gear guides, etc.) as well as fastening devices or brakes with which the pivot movements or longitudinal movements can be established and/or can be brought to preset positions via adjustable locking devices 17cd, 17ce, 17cg, 17ch or snap-in devices and/or electronically controlled and their movements adjusted to each other. For example, an adjustment of the extension guide 17l, 17m, 17ma, 13da-13do on the desired extension path can be conducted in this way or in the system axis pivot devices adjustment to the desired anteversion and inclination angle 5, by snapping in the system axis pivot devices in a certain pivot angle. Especially for milling of the pear-shaped shoulder joint socket 9b a program-controlled cooperation of the drive of the system axis pivot devices 17b-17l, 7n and possibly linear guide 13vc-13ve of the slide 13va with the drive motor 13vb mounted on it can be advantageous for a finger milling cutter or similarly-shaped milling cutter (the milling cutter is then preferably provided with the milling edges on the front and flanks).

Exact fixation of the shoulder 9 or pelvis 11 and therefore the corresponding joint socket 9b, 11a is achieved in that the parts of the pelvic bone or shoulder blade 9a and/or the clavicle 9h and/or parts of the upper thorax 6 are acted upon by the holding devices 14-16, 7a-7w at the locations that only have a thin soft tissue cover and therefore little flexibility and overall do not permit displacements or rotation or torsion of the pelvis 11 or shoulder 9.

In the pelvis 11 the following holding devices adapted or adaptable to the corresponding bone contours can be applied to the following body parts, whereby these holding devices can be combined with each other and/or designed in one or multiple parts:
An ischium-pubic bone holder 15 preferably adjustable transversely and longitudinally relative to the operating table axis with a possibly height-adjustable and pivotable ischium-pubic bone pad 15l supports the ischium and pubic bone 11k, 11l from the bottom (referred to the patient) and partially from the inside of the ischium branch. The ischium-pubic bone holder 15 or its support part 15c can be guided via a mechanism that produces its center position between the lateral pelvic supports 14x (for example, two or preferably four strips that are mounted to pivot on the lateral pelvic supports 14x or their holders 14j and on the support part 15c of the ischium-pubic bone holder 15). The support part 15c can also serve as center part of the rear pelvic brace 14i, 14h and/or as support for the lateral pelvic supports 14x, 14zl or their holder 14j or their foot plate 14f.
A symphysis holder 15n-15s with symphysis support 15v supports the symphysis 11j from the front and can be mounted height-adjustable and pivotable on the front pelvic brace 16 or on the ischium-pubic bone holder 15 or its foot part 15c, as required.
Lateral pelvic supports 14j, 14m, 14x, 14xa, 14zl support the pelvis roughly in the area of the iliac crest 11i from the sides. They consist of one or two solid or semirigid (for example, sheathed spring steel sheet) or flexible plates or belts on each side. They are optionally also mounted height-adjustable, transversely and longitudinally adjustable on the foot plate 14f, the holders for the lateral pelvic support 14j on the base 14d of the pelvic fixation, on one of the holding devices 14-16, on the support part 15c of the ischium-pubic bone holder 15 and/or on the front pelvic brace 16. Semirigid plates can also be acted upon by pivotable support arms 14zc, 14zf, which are mounted longitudinally and preferably transversely adjustable and possibly pivotable on the base 14d or front pelvic brace 16. A favorable design consists of the fact that the front 14xa and rear lateral pelvic supports 14x are relatively rigid at the base and increase in elasticity starting from it so that especially the part at which they overlap is more flexible. The flexible, strip-like lateral pelvic supports 14zl can be combined on the rear support 14zm with those of the iliac crest holder 14r (see FIG. 16). During back supporting of the patient the front holder or tightening devices 14zn are preferably designed so that a clamping device is provided for holding, which then permits tightening of the lateral pelvic support 14zl through a longitudinal guide, pivot guide or by rolling.
Iliac crest holders 14p, 14s support the iliac crest 11i from the top and can be designed as strips with pads 14p or as semielastic to elastic bands 14s, which can be shaped in cross section so that they lie on the iliac crest and are bent downward on the outside in order to encompass the iliac crest and/or angled upward on the inside. They can be mounted directly or in a tightening device 14t on the lateral pelvic supports 14m, 14x, 14xa or on an adjustable or pivotable holder of the lateral pelvic supports 14j or on a foot plate of the lateral pelvic support 14f, on the rear pelvic brace 14i, on the support part 15c of the ischium-pubic bone holder 15 and/or on the front pelvic brace 16. The iliac crest holder 15s and lateral pelvic support 14xl can have a common origin (for example, the foot plate of the lateral pelvic support 14f) and/or the lateral pelvic support 14xl can be mounted on the rear part of the iliac crest holder 14s (see FIG. 16).
A rear pelvic brace 14i supports the pelvis 11 from the rear and can consist of one or more parts, in which they consist of, for example, one or two separate ischial tuberosity supports 14h, one or two rear iliac bone supports and possibly a sacrum support and a support for the lower lumbar spine. The ischium-pubic bone holder 15, its support part 15c or its pad 15l, the lateral pelvic supports 14x, their holders 14j or their foot part 14f can partially or fully assume the functions of the rear pelvic brace 14i. The rear pelvic brace can also consist of additional holders that are mounted adjustable on the base 14d of the pelvic fixation or on the system center adjustment devices 2-2d, 17b.
Front upper iliac bone holders 14q, 14w support the iliac bone 11g from the front, in which they can be combined with the iliac crest holders 14s, 14p and/or the lateral pelvic supports 14m, 14x, 14xa, 14zl or mounted separately on the front pelvic brace 16 or on the lateral pelvic supports 14x, 14xa, 14m. During support on the front pelvic brace 16 they are preferably connected via a connection, for example, a stud bolt 14v, to the iliac crest holders 14s or their support or tightening device 14t and/or the support and tightening device of the band-like lateral pelvic supports 14zn and adjustable laterally together with them.
A front pelvic brace 16 supports the pelvis 11 from the front and can consist of one or more parts connected to each other and preferably adjustable relative to each other, for example, a lower part 16j with a symphysis support 15v preferably adjustable longitudinally and possibly in height, and an upper part 16b. This has transverse longitudinal guides 16o and/or a transversely movable strip 16e with transverse longitudinal guides 16d. The following can be mounted in the transverse longitudinal guides 16d, 16o: the front upper iliac bone holders 14w, 14q, the lateral pelvic supports 14m, 14x, 14xa, 14zl and their support arm 14zf and the iliac crest holder 14p, 14s and its tightening device 14t-14v, in which the latter is preferably mounted on the top, and/or a plate 16p with the other devices preferably on the bottom and connected, for example, by a stud bolt 14v to the tightening device 14t-14v. The following can be mounted on the plate 16p: the iliac bone holder, the iliac crest holder 14s, 14p, the lateral pelvic supports 14m, 14x, 14xa, 14zl and their support arm 14zf. The front pelvic brace 16 can have connection devices (for example, strips, rods, stud bolts, belts) to the following devices: the ischium-pubic bone holder 15 or its pad 15l, its support bar 15c, the symphysis holder 15n-15s and/or the lateral pelvic supports 14j, 14m, 14x, 14xa, their holder 14j, their foot plate 14f or a holder 14y of the front pelvic brace 16. The front pelvic brace 16 is mounted adjustable in height and possibly laterally and/or longitudinally on these connections. The connections on the upper part and in the upper area preferably occur in one of the transverse longitudinal guides 16d, 16o or by means of connection structures 16f in their extension.
In addition to the aforementioned suspensions, the following devices can be mounted on the base of the fixation device 14d, on a system center adjustment device 2-2d, 17b, 17c, 17l, 7n or a connection to the operating table 1a or possibly to the rear pelvic brace 14i: the ischium-pelvic bone holder 15, its foot part 15g or support part 15c, the lateral pelvic supports 14j, 14m, 14x, 14xa, 14zl or their holder 14j, their foot part 14f, their support arms 14zc, 14zf, their rear suspension 14zm, the holder 14y of the front pelvic brace 16, the rear pelvic brace 14i and at least the rear suspension of the iliac crest holder 14r.
The transitions and parts of the rear pelvic brace 14i, possibly including the lateral pelvic supports 14m, 14x, 14xa, can be covered by a semirigid plate or shell 14zi, which is preferably shaped according to the base.
The holding devices 14f-14zn, 15, 16, 7d-7m, 7p-7u of the pelvis and the shoulder can be provided with cushions 14zk and especially with vacuum cushions or cushions corresponding to a vacuum mattress 14zk and designed partly as belts (also belts profiled in cross section).
All holding devices 14f-14zn, 15, 16, 7d-7m, 7p-7u of the pelvis and the shoulder are preferably mounted in longitudinal guides and/or pivot guides arranged so that the holding device can be adjusted roughly in the direction in which it acts on the bones.

FIGS. 9 and 10

The bony pelvis consists of the wing of the ilium 11f, the sacrum 12b and lower lumbar spine 12a, the anterior superior iliac spine 11g, the symphysis 11j, the pubic bone 11k and the ischium 11l. 11a represents the acetabulum.

The base 14d of the fixation device 14-16 of the pelvis in the axis of the operating table 1 is mounted to move on the connection 1a (a strip) to the operating table, which has a longitudinal guide 2a with the function of a system center adjustment device 2, and mounted fixable by the clamping screw 14e.

The ischium-pubic bone support 15j has a cushion similar to a bicycle saddle, the ischium-pubic bone pad 15l.

The sacrum 12b and the posterior parts of the hip including the posterior iliac spine 11h lie on the rear pelvic brace 14i.

FIG. 9 shows a practical example of the fixation device in a transparent view from the side, in which no front pelvic support 16 is provided. A base plate 15a of the ischium-pubic bone holder 15 is mounted adjustable on the connection 1a to the operating table. The rear ischial tuberosity support 14h, a pad for support of the ischial tuberosity 11m and an ischium-pubic bone support 15j designed as a hollow cylinder are fastened on it, which guides a symphysis holder 15n designed in cross section, for example, as a round, square or hexagonal rod. On the upper end this has an angled arm 15s and carries on the end a disk-like widening with cushion, the symphysis support 15v with a pad. In the base plate 15a of the ischium-pubic bone holder 15 a stud bolt with wing nut 15t is anchored, which forces the rod and therefore the symphysis support 15v downward or onto the symphysis. The base of the pelvic fixation 14f supports in transverse longitudinal guides 14c the foot plate 14f of the holder 14j of the lateral pelvic support 14m. The holder 14j of the lateral pelvic support contains in the upper part two longitudinal guides 14k (simple elongated holes in this practical example), in which a plate, the lateral pelvic support 14m is mounted, which again supports a pivot plate 14n with pivot guide 14o. The iliac crest holder 14p with the anterior iliac spine holder 14q is mounted on the pivot plate 14n. The iliac crest holder 14p consists of a cushioned pad strip adapted to the iliac crest with the anterior superior iliac spine holders 14q.

FIG. 10 shows a practical example of a pelvic fixation device in a transparent view from the side in which the symphysis support 15v, the front braces or clamping devices 14t of the band-like iliac crest holder 14s and the anterior superior iliac spine holder 14w are mounted adjustable on the plate 16a of a front pelvic brace 16. This front pelvic brace 16 is adjustable in height on connection strips 15q of the ischium-pubic bone holder 15 and mounted on a holder 14y of the front pelvic brace, which is fastened in an anchor 14ya in the base 14d of the fixation device. The base 14d of the fixation device in this example is made in one part and the foot part 15g of the ischium-pubic bone holder 15 is adjustable in length in the support part 15c of the ischium-pubic bone holder 15. The support part 15c is mounted transversely movable on the base 14d. A cross member 15p is situated on the ischium-pubic bone support 15k, on whose outside the two arms 15q of the symphysis holder 15n (here designed as stud bolts with thread) are mounted for a height-adjustable connection to the front pelvic brace 16. They hold in connection structures 16n, for example, in longitudinal guides or elongated holes, the lateral protrusions 16m of the front pelvic brace 16a. The front pelvic brace plate 16a has an elongated hole 16j in the center, in which the stud bolt 15r of the symphysis support 15v is mounted with a fastening screw. The rear ischial tuberosity support 14h is mounted on the base 14d. The rear lateral pelvic supports 14xm are mounted on the foot part 14f, which is mounted to move transversely in the base 14d of the pelvic fixation. The front lateral pelvic supports 14xa are mounted on a plate 16p adjustable transversely on the front pelvic brace 16. The front pelvic brace 16a has in the upper part an elongated hole across the longitudinal axis of the operating table. In it there is a stud bolt with wing nut 14z, which is fastened as a holder of the front pelvic brace 14y in the base 14d with an anchor of the holder 14ya. In a second transverse elongated hole 16d the plate 16p is mounted and guided with the anterior iliac spine holder 14w, the front lateral pelvic support 14xa with a stud bolt 14v. Above the elongated hole on the stud bolt 14v the holder 14t and the tightening device of the band-like iliac crest holder 14s is mounted with an idler 14u. The rear support 14r of the iliac crest holder 14s is mounted on the sacrum-ilium support 14i.

FIG. 11 shows a view from the foot side of the patient of an ischium-pubic bone holder 15 with ischium-pubic support 15j and pad 15l. The symphysis holder 15n is mounted height adjustably in the ischium-pubic bone support 15j, which is split into two arms 15q and whose height adjustment occurs via a stud bolt in the ischium-pubic bone support 15j and a tension screw 15u. The arms 15q of the symphysis holder 15n are continued in the horizontal arm 15s on which the symphysis support with pad 15v is mounted.

FIG. 12 shows a view from the side of an ischium-pubic bone holder 15 whose support part 15c is mounted on round rods 14d (which serve as longitudinal guides) of base 14d of the fixation device 14-16 or a system center adjustment device 2-2d, 17b, 17c, 17l, 7n transversely movable and whose foot part 15g is mounted longitudinally movable in a square tube 15f of the support part 15c and can be fastened by the screw 15i in the clamping wedge 15h. (The square tube 15f of the support part 15c is slit in the longitudinal direction in the area of the clamping wedge.) The symphysis holder 15n is again mounted in the ischium-pubic bone support 15k adjustable in height and rotationally stable with the screw 15m. The symphysis holder in this practical example has a bent arm 15o.

FIG. 13 shows a transparent view from the side of an ischium-pubic bone support 15k on which the pad 15l is supported adjustable in height and pivotable (pivot bearing 15m). A shaft or cylinder 15mb is inserted into the ischium-pubic bone support 15k, which has a threaded hole through which the adjustment screw 15md pivots the pad 15l.

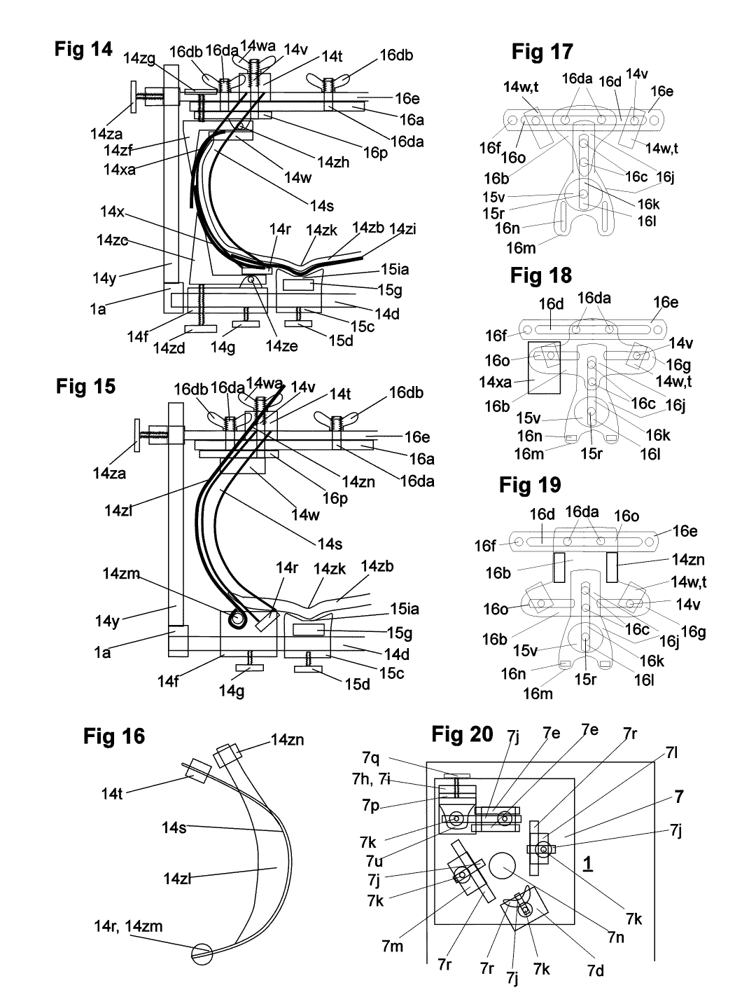

FIG. 14 shows a cross section through one half of a pelvic fixation device with semirigid lateral pelvic supports 14x, 14xa and a band-like iliac crest holder 14s. A strip 14a is connected to the surgical table 1. The base 14d of the pelvic fixation is mounted in it with transverse strips serving as longitudinal guide. The support part 15c of the ischium-pubic bone holder 15 is adjusted by the clamping screw 15d to the strip of the base 14d. The support part 15c supports the foot strip 15g of the ischium-pubic bone holder 15. In this example the support part 15c serves as part of the rear pelvic brace 14i, especially the spinous processes of the lower lumbar spine 12a and the sacrum 12b. For this purpose it has a countersinking 15ia running in the longitudinal direction. On the same strip of the base 14d the foot plate 14f of the lateral pelvic support 14x is mounted adjustable by the clamping screw 14g. It has a protrusion with axial shaft 14ze as support of the support arm 14zc, which is pivoted by the adjustment screw 14zd and which supports the semirigid lower lateral pelvic support 14x. The lateral pelvic support 14x and the rear suspension 14r of the iliac crest holder 14s is mounted on the support arm 14zc. The transverse strip 16e is mounted laterally adjustable on the plate 16a of the front pelvic brace in an elongated hole guide with the threaded pins 16da and screws 16db. The front pelvic brace 16 is mounted with the strip 16e with the clamping screw 14za on the holder 14y of the front pelvic brace adjustable in height. In another transverse elongated hole guide the stud bolt 14v is fastened in the plate 16p. A tightening device 14t of the iliac crest holder 14s is adjusted laterally movable with the screw 14wa on the stud bolt 14v. The support arm 14zf of the upper lateral pelvic support 14xa is also mounted to pivot in the pivot bearing 14zh on the plate 16p on the bottom of the front pelvic brace 16. The front iliac bone spine holder 14w is also mounted on the support arm. The support arm is pivoted by the screw 14zg. The parts of the lateral pelvic support 14x serving as parts of the rear pelvic brace 14i and of the support part 15c of the ischium-pubic bone holder 15 are covered by a semirigid cover plate 14zi. This plate as well as the lateral pelvic supports 14x and 14xa are also covered with a vacuum mattress-like cushion layer 14zb.

FIG. 15 corresponds to FIG. 14, but with a continuous semirigid or flexible lateral pelvic support 14zl and without support arms of the lateral pelvic support without the semirigid cover plate 14zi. The vacuum cushion 14zb here is restricted to the rear area of the lateral pelvic support 14zl. The rear support 14zm of the lateral pelvic support 14zl as well as the rear support 14r of the iliac crest holder 14s are situated on the foot plate 14f of the lateral pelvic support 14zl. The plate 16p in this practical example only carries the anterior iliac spine holder 14w. The iliac crest holder 14s is mounted in tightening device 14t as in FIG. 14. Beneath it a fastening and tightening device 14zn is situated for the lateral pelvic support 14zl.

FIG. 16 shows a practical example in which the iliac crest holder 14s (narrow side, since it is a side view) and lateral pelvic support 14zl are together fastened in a rear receptacle 14r, 14zm. The lateral pelvic support 14zl is fastened in the rear to maximum middle part of the device on the iliac crest holder 14s.

FIG. 17 to FIG. 19 show front pelvic braces 16 as seen from above. They consist of a plate 16a or of two plates connected to each other and adjustable in a longitudinal guide (for example, two stud bolts 16c in an elongated hole 16k)—an upper plate 16b and a lower one 16j. In the one plate 16a or in the upper plate 16b a transverse longitudinal guide 16o and/or a transverse longitudinal guide is situated in a protrusion 16g (for example, elongated hole 16d with two stud bolts 16da). In one of the transverse longitudinal guides 16o, 16d, preferably on the bottom of the plate 16a, 16j or the strips 16e, a plate 16p is mounted with the front upper iliac bone spine holders 14w, possibly the holders 14zn of the lateral pelvic supports 14m, 14xa, 14zl, their support arms 14zf, 14zc with a stud bolt 14v. On the top of the plate 16a the holding device or tightening device 14t of the iliac crest holder 14s is mounted on this stud bolt and possibly the holder or tightening device 14zn for the flexible lateral pelvic support 14zl. In one of the transverse longitudinal guides 16o, 16d or the strip 16e with transverse longitudinal guide 16d, 16o or in the connection structures 16f or holes on the end of the transverse strip 16e a holder 14y of the front pelvic brace (for example, rods, stud bolts) or a holder of the lateral pelvic supports 14m, 14x or their holders 14j are adjustably supported.

On the side of the hip undergoing surgery the connection to the holder of the front pelvic brace 14y is preferably produced by the lateral connection structure 16f, whereas the holder of the front pelvic brace 14y comes to lie on the other side in the elongated hole 16d, 16o. Because of this an overhang of the strip on the side undergoing surgery is avoided (which could interfere during the operation). On the one plate 16a or the lower plate 16j of the front pelvic brace the plate has an elongated hole 16l in the longitudinal direction of the operating table for a stud bolt 15r of the symphysis support 15v. Laterally it preferably has two lateral protrusions 16m with two connection devices 16n (for example, elongated holes) in which the arms 15q of the symphysis holder are accommodated or connections to the ischium-pubic bone holder 15.

In FIG. 17 the front pelvic brace consists of a plate (16a).

In FIG. 18 the front pelvic brace consists of two plates (16g, 16j). FIG. 18 largely corresponds to FIG. 17 except that the transversely mounted strip 16e only serves to accommodate the holder of the front pelvic brace 14y or connections to the lateral pelvic supports 14x or the upper holders of the front pelvic brace. For this purpose the upper plate 16b has two lateral protrusions 16g, which contain on each side a transverse elongated hole 16o in which the anterior superior iliac spine holder 14w and the iliac crest holder 14t are supported with stud bolts 14v, etc. Instead of elongated holes, laterally beneath the symphysis support this practical example has two braces or tightening devices 16n for belts, bands, cables, etc. with which it is connected to the symphysis support holder, the pubic bone support, etc.

In FIG. 19 the holder and tightening devices 14zn of the flexible lateral pelvic supports 14zl are also mounted.

In the shoulder 9 for fixation, in addition to belts 7a over the thorax 6 and/or belts 7b over the shoulder(s) (which also can carry adjustable clavicle pads 7c), some of the following body parts are preferably held by the holding devices of a shoulder fixation device: the clavicle 9h, the shoulder blade 9a, especially the angulus inferior 9g of the shoulder blade, the coracoid process 9e, the acromion 9f and possibly the spina scapulae 9d and possibly the lateral and/or medial shoulder blade edges. For problem cases a screw 7f can also be screwed into the spina scapulae 9d or the dorsal acromion 9f, which is accommodated in a screw receptacle 7g of the base 7 or the support plate 7o and contributes to fixation. The holding devices 7d-7m, 7p-7u are preferably mounted on a support plate 7o. This is positioned on the shoulder blade 9a, for instance, with the patient sitting, the holding devices 7d-7m, 7p-7u are mounted, whereupon the patient is brought into a horizontal position and the support 7o fastened with one or more connections 7n (preferably snap-in connections) to the base 7 and the belts 7a, 7b applied. This connection 7n can also be formed so that it additionally serves as pivot device or combined pivot longitudinal adjustment device.

The base 7 of the shoulder fixation is supported relative to the system center adjustment devices 2-2d, 17b, 17c, 17l, 7n and the system axis pivot devices 17b-17l, 7n according to the specifications of the pelvic fixation device 14-16 or connected to it or integrated in it. Functions of the system center adjustment device 2-2d, 17b, 17c, 17l, 7n and possibly the system axis pivot devices 17b-17l, 7n (for example, the connection device 7n of the support plate 7o with the base 7) can also be integrated as in the fixation device 14-16 of the pelvis in the holding devices 7d-7m, 7p-7u of the shoulder. The most important devices of the shoulder blade in principle are: a holding device 7h, 7ha, 7i, 7p, 7q, 7s-7u functioning similar to a gripper for grasping the acromion 9f and the coracoid process 9e. It has two claw-like or hook-like curved strips 7u, 7ha or pads, the front one to hold the coracoid process 9e from the front, the transition 7s to act on the area of the acromion 9f on the head side and its rear claw 7ha to enclose the dorsal edge of the acromion 9f, including the transition to the spina scapulae 9d. The two claws are connected to each other by adjustment devices 7k, 7t and/or pivot devices 7p, 7q. The holding device can consist of one part and be mounted fixed or movable on the support plate 7o of the holding devices or the base 7 or both parts are mounted separately and are adjustable in adjustment devices relative to each other in the support plate 7a or the base 7. The second base holding device exists in a holder 7d, 7r of the angulus inferior scapulae 9g, which is mounted adjustable on the support plate 7a or the base 7. The aforementioned holding devices 7h, 7ha, 7i, 7p, 7q, 7s-7u can also be provided.

FIGS. 20 to 22 show practical examples of the fixation device of the shoulder.

FIG. 20 shows a view of the operating table 1 and the base 7, in which no support plate 7a is present in this practical example. The base 7 is pivotable and adjustable in height via a connection 7n on the operating table 1. The holding devices are mounted on the base 7: the holder of the acromion 7h and the coracoid process 7i, the arc-like or claw-like pad 7u holding the coracoid process 9e, the pivot axis 7p of the holder 7t of the coracoid process 9e, the adjustment screw 7q of the pivot device 7p, the holding device 7m of the lateral shoulder blade edge, a holder 7d of the angulus inferior scapulae 9g, a holding device 7l of the medial scapula edge each with the corresponding pad strips 7r and two holders with pad strips 7e for holding the spina scapulae. The holding devices are supported fixed in longitudinal guides 7j (in this example elongated hole guides) on the base 7 with fastening screws 7k.

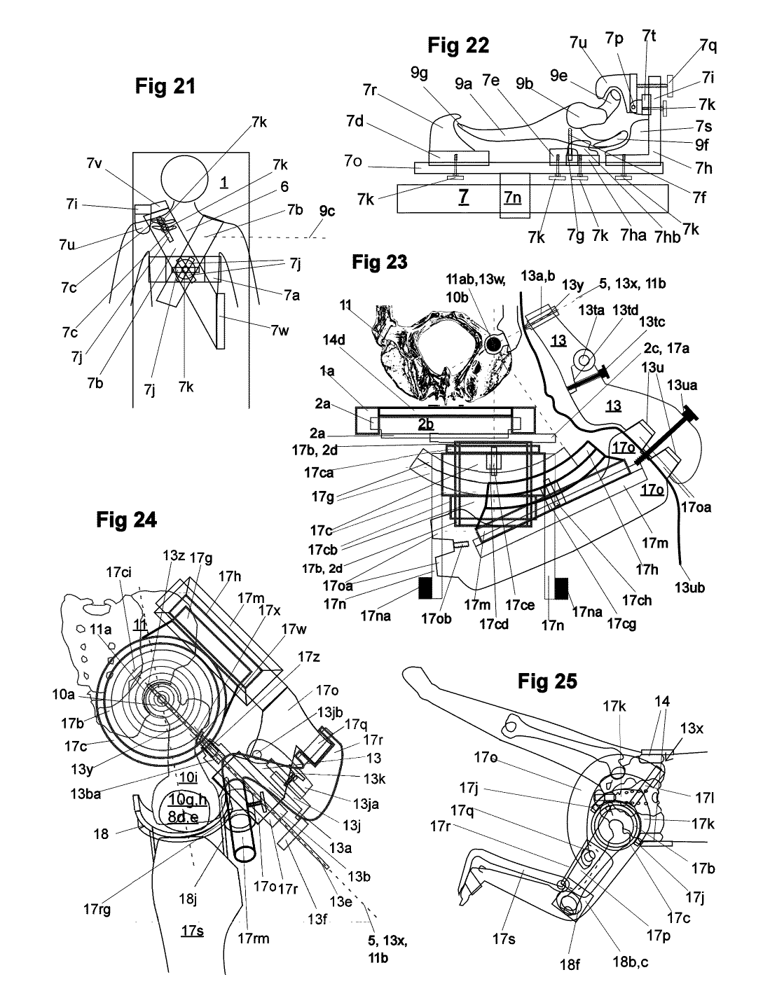

FIG. 21 shows a view of an operating table 1 with the patient positioned on it, whose thorax and shoulder are fixed with a thorax belt 7a and shoulder belts 7b. The shoulder belt is fastened here on the operating table 1 with the brace 7w and with a belt brace 7v on the acromion-coracoid holder 7i or on the support plate 7o or the base 7. Two clavicle holders with pads 7c are mounted adjustable in the longitudinal guides 7j (elongated hole guides) with fastening screws 7k. The thorax belt 7a and the shoulder belt 7b are adjustable in elongated hole guides 7j with the fastening screws 7k.

FIG. 22 shows a transparent view from the head side of the patient of a shoulder blade 9a with a shoulder fixation device. In contrast to FIGS. 20 and 21, the holding devices 7d-7m, 7p-7u in this practical example are supported on a support plate 7o, which is mounted on the base 7 via a snap-in device 7n. The shoulder blade 9a ends in the angulus inferior 9g. Laterally the shoulder blade continues forward into the coracoid process 9e and rearward as a continuation of the spina scapulae in acromion 9f, which is cut transversely in this drawing so that the part running forward and up is not drawn. 9b is the shoulder joint socket. The upper acromion holding plate 7h and the holder of the coracoid process 7i in this practical example consist of an angled plate. A support plate 7t with a pivot bearing 7p is mounted on it adjustable in height, on which the claw-like pad 7u is supported, which holds the coracoid process 9e. Pivoting occurs through the adjustment screw 7q. The acromion holders in this practical example consist of two parts, a medial holding claw 7hb, which is mounted on a plate 7ha adjustable by the screw 7k, as well as a pad 7s, which the acromion acts on from the head side of the patient, from the side in the drawing. In addition, a holder with pad 7e for the spina scapula and an adjustably positioned holding plate 7d with a pad 7r to hold the angulus inferior are additionally shown in the drawing.

The extension device 17-18 has an extension arm 17o, which accommodates the extremity with an extremity extension brace 17p, 17w-17z, 17za-17zr and with the extension guide 17l, 17m, 17ma, 13da-13do extends the joint head from the socket and then pivots it with the system axis pivot devices 17b-17l, 7n in order to obtain the required joint gap.

The system axis pivot devices 17b-17l, 7n serve to adjust the system axis 13x, i.e., the direction of extension guide 17l, 17m, 17ma, 13da-13do (and if the control device 13 is mounted on the extension arm 17o or forms a unit with it, also the axis of the tool guide 13a) to the planned anteversion and inclination angle (for example, by means of angle scales). The axes of at least one, preferably all system axis pivot devices 17b-17l, 7n run through the system center 13w. System axis pivot devices can also be combined with the extension guide 17l, 17m, 17ma, 13da-13do, for example, in which they consist of a pivot axis 17l, 17ma on which the extension arm 17o is mounted to pivot and can be moved linearly and coaxially.

The extremity holder 17r with its adjustment devices and pivot devices 17q, 17ra-17rg, 17ri, 17rj, 17rm, 18, 18a-18i guarantees adjustment of the arm or leg to the system axis 13x true to angle, for example, to make the system bore 13y. It can be adjustable in length, fixed on the extension arm 17o, mounted adjustable with an insertion or snap-in connection 17m with at least one pivot device 17q or a length adjustment. It can have pivot devices 17ra, 17rc, 17rf-17rh, 17rj, 17rm and/or arc guides 17rd, 18a-18c, 18h and/or longitudinal guides 17rb, 17rg, 17rh, 17rm, 18e, on which the at least one support shell 17s for the extremity is supported. A pivot guide with a length adjustment can then be combined in which the support 17rm allows both a pivot movement and an axial displacement. If the extremity extension brace 17p, 17w-17z, 17za-17zr is applied to the soft tissues of the extremity, it can be mounted adjustable on the extremity brace 17r, otherwise preferably on the extension arm 17o or on the control device (if this is mounted on the extension arm or forms a unit with it).

In the area of the system center-adjustment devices 2-2d, 17b, 17c, 17l, 7n of the system axis pivot devices 17b-17l, 7n, on the extension arm 17o or on the control device 13 x-ray templates 13n, 17ci, 17u with line structures (for example, parallel lines) 13r and/or centering structures 13s (for example, concentric circles) can be mounted, which serve by means of a C-arc x-ray device to adjust the system center 13w to the joint center point 8b, 11ab and to adjust the extension guide 17l, 17m, 17ma, 13da-13do and the tool guide 13a of the control device 13 (or system axis 13x) to the planned inclination and anteversion angle 5.

FIGS. 23 to 30 show practical examples of extension devices 17 and system axis pivot devices 17b-17l for the hip.

FIG. 23 shows a practical example of an extension device in which the base 14d of the pelvic fixation device 14 is mounted fixed on the connection devices 1a to the operating table 1. A system center adjustment device 2b movable lengthwise in a longitudinal guide 2a of a connection 1a to the surgical table 1 houses in the longitudinal guide 2a a plate as a transversely movable system center adjustment device 2c, which forms the base 17a of the extension device. A hollow cylinder 17b is fastened to the base 17a, on which a pivot cylinder 17c is mounted to pivot and is adjustable in height and therefore has the function of a system center adjustment device 2d adjustable in the vertical direction in addition to the function of system axis pivot device. The height adjustment occurs through a screw cylinder 17cb, which is supported on the pivot cylinder 17b with a screw thread. A ring 17ca is situated above the pivot cylinder 17c, which is adjustable upward and can be fastened as axial guide. It has a snap-in device (recess) for a locking pin 17ce, which is mounted in a support 17cd on the pivot cylinder 17c. An arc guide 17g is fastened to the pivot cylinder 17c. Its axis runs horizontally and its center point lies in the system center 13w. The supported arc 17h is supported on it. Here again a snap-in operation 17cg with a locking pin 17ch is applied which permits snapping in at a preset pivot angle. The extension guide 17m is mounted on the support arc 17h, which is formed here as a longitudinal guide. The extension arm 17o is fastened to it or forms a unit with it. The extension arm 17o has two snap-in devices 17oa in the form of conical pins, on which the control device 13 is mounted and locked by means of corresponding recesses 13u and a locking screw 13ua. The control device 13 has a pivot axis 13ta which makes it possible to pivot the upper part of the control device away from the extremity and make it accessible. In order to ensure exact positioning of the control device, a stop 13td and a locking screw 13tc are provided. The control device carries the tool guide 13a, whose axis coincides with the system axis 13y. Holding strips 17n with snap-in devices 17na for a C-arc x-ray device are mounted on both sides on the pivot cylinder 17c.

FIG. 24 shows a transparent view of an extension device. A template 17ci with concentric circles is situated in the horizontal plane in the hollow cylinder pivot support 17b, 17c with vertical axis. The extension arm 17o is mounted on the horizontal arc guide 17h, 17g via the extension guide 17m. It has a fixed connection with the control device 13, i.e., the control device and extension arm consist of one unit. The tool guide 13a supports a bushing 13b on whose front end a recess 13ba with thread on a connection 17z (threaded pin) the extension plate 17w is screwed. The extension plate 17w is screwed with bone screws 17x to the femur 10i. The drill 13e of the system bore 13y with a measurement head 13f is situated in the tool guide 13b. The system bore has a lengthened bore 13z in the acetabulum 11a. A control device 13 has a measurement device 13ja in a pivot bearing 13jb with a measurement strip 13j adjustable by the screw 13k. The extension arm 17o supports the extremity holder 17r in the pivot guide 17q. This has a rod 17rm on which a body 17rg is mounted to pivot and is adjustable lengthwise. It carries an arc guide 18 on the distal femur, which again holds the lower leg support shell 17s via strips 18j.

FIG. 25 shows a transparent view from the top of an extension device with system axis pivot devices 17b-17l. The hollow cylinder system axis pivot device 17b, 17c corresponds to those of FIGS. 23 and 24. Instead of arc guide 17g, 17h with a horizontal axis, however, it has two pivot supports 17j on which a holder 17k is mounted. A combined pivot bearing and extension guide 17l is mounted on it, on which the extension arm 17o is supported. Thus in this practical example both a pivot device 17b, 17c with a system center adjustment device 2 (height adjustment) and a pivot device 17l with extension guide 17l are combined. The extension arm 17o carries an extremity brace 17r-17t on which the extremity extension brace is mounted in the form of a shell 17p.

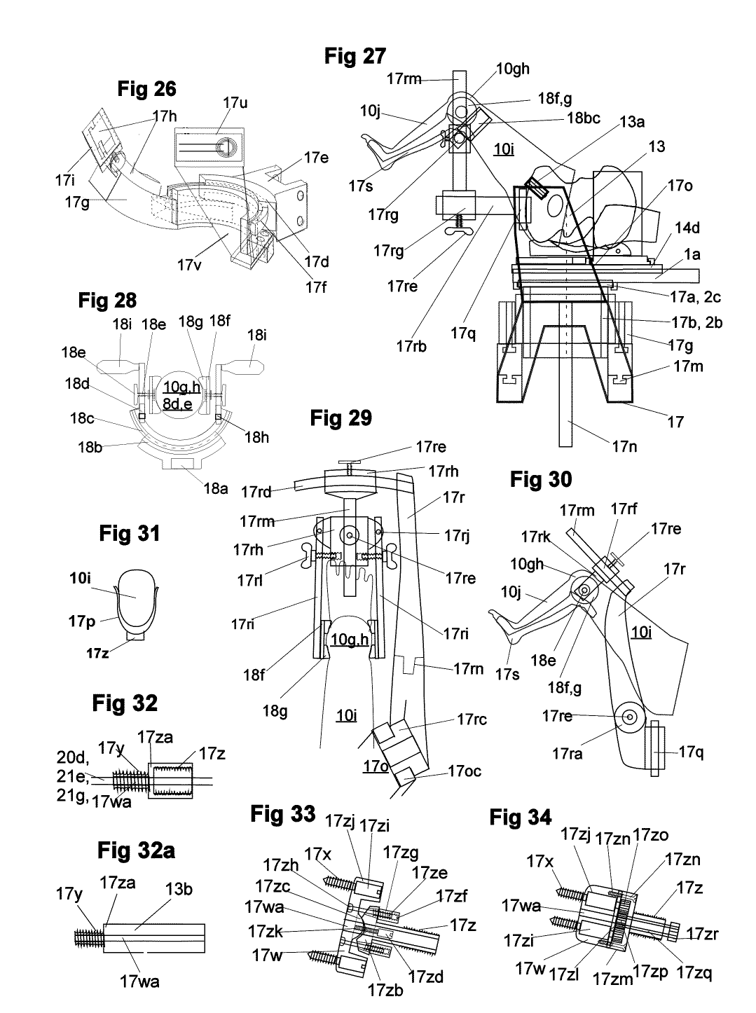

FIG. 26 shows system axis pivot devices, the first of which consists of an arc guide 17d, 17f with vertical axis and the second of an arc guide 17g, 17h with a horizontal axis, in which the axes of the two arc guides intersect at the system center 13w. The holder 17e is preferably mounted on a device adjustable in height on the system center adjustment devices 2a-2d, 17b, 17c, 7n. A holder 17v for an x-ray template 17u is situated on the guided arc 17f of the first arc guide to adjust the system center to the center point of the joint. 17i is an adjustable stop on the guided arc 17h of the second horizontal arc guide 17g.

The extremity holder 17r is mounted fixed or mountable or longitudinally adjustable or via one to two pivot devices on the extension arm 17o or supported on it. It can form a unit with the extension arm 16o. It can have adjustment devices 17rb, 17rg, 17rh, 17rm, 18e (for example, length adjustments) and pivot devices 17q, 17ra, 17rc, 17rd, 17rf-17rh, 17rj, 17rm, 18, 18a-18c, 18h. They serve both for adjustment to different leg sizes and for adjustment of the joint axis of the humerus 8c or femur 10f to the desired inclination and anteversion angle 5 (for introduction of the system bore 13y and for guiding of the milling cutter shafts 19b, 19m, 20d, 20r and press-in/-on rods 21e, 22m of the prosthesis shells 22a, 21h). For this purpose pivoting around two pivot axes is necessary. A pivot axis, which runs roughly through the middle of the knee or elbow joint and the system center is particularly favorable (and therefore after making the adjustment of the system center adjustment devices 2-2d, 17b, 17c, 17l, 7n through the joint center point). This can occur through a pivot guide 17rm or an arc guide 18 on the distal femur or humerus. The extremity holder 17r supports a lower leg or forearm support shell 17s and, if required, also a support shell for the distal femur or humerus. In order for the adjustment to remain as exact as possible, braces in the area of the knee or elbow for the epicondyles of the humerus 8d or femur 10g are favorable, since here the bone lies directly beneath the skin. This is accomplished in the practical examples by epicondyle holders 18f with pads 18g, which are pressed onto the epicondyles by pivot strips 17ri or screw guides 18e.

One possible support of the extremity holder 17r on the extension arm 17o also consists of the fact that an annular support 170c is mounted in the direction of the system axis 13x on the extension arm 17o in which an annular support 17rc of the extremity brace 17r is coaxially accommodated. The central coaxial recess of the annular support of the extremity holder then preferably assumes the function of the control device or tool guide.

FIG. 27 shows a transparent view from the side of an extension rail 17 with extremity holder 17r (as well as connections 1a to the operating table 1, system center adjustment devices 2, system axis pivot devices 17b, 17c, 17g-17k and an extension guide 17m according to FIGS. 10, 23, 24, 25). The extremity holder 17r is supported in a pivot bearing 17q on the extension arm 17o (which forms a unit here with the control device 13). The body 17rg is supported adjustable lengthwise on the linear guide (strip) 17rb with the fastening screw 17re. The support body 17rg carries a rod 17rm, on which an additional support body 17rg is supported to pivot and is adjustable in height. This again supports the arc guide 18 on the distal femur.

FIG. 28 shows a cross section through an arc guide 18 of the extremity holder 17r on the distal femur or humerus roughly at the height of the knee 10h or elbow joint 8e with epicondyles 8d, 10g. 18b is the arc supported on the extremity holder 17r with the brace 8a, which supports the second arc 17c. Two lateral plates 18d are mounted on it, in which the epicondyle plates 18f with pads 18g are guided in screw guides 18e and which accommodate the femur or humerus epicondyles 10g. In the lateral plates 18d supports 18h are found for pivotable support of the lower leg or forearm support shell 17s. 18i are handles on the lateral plates to perform the adjustment.

FIG. 29 shows an extremity holder 17r, which is supported with an annular pivot bearing 17rc in an also annular pivot bearing 17oc on the extension arm 17o. (The axial direction here preferably lies on the system axis 13x and runs through the system center 13w.) The extremity holder 17r also has a braceing or snap-in device 17m. It carries an arc guide 17rd, which adjustably supports an epicondyle holder suspension body 17rh with the fastening screw 17re. The center of the arc guide preferably coincides with the system center 13w. The epicondyle holder-suspension body 17rh has a rod 17rm with longitudinal and pivot guide. This supports epicondyle pivot strips adjustable in the pivot during 17rj and by adjustment screws 17ri, which carry the epicondyle holders 18f with pads 18g. The axis of the rod 17rm preferably runs through the center of the knee joint 10h and the system center 13w.

FIG. 30 shows a transparent view from the side of an extremity holder 17r. It is supported with a vertical pivot bearing 17q on the extension arm 17o. A second pivot bearing 17ra with horizontal axis can be locked by a fastening screw 17re. The extremity holder 17 carries a rod 17rm as longitudinal and pivot guide 17rm for an epicondyle support body 17rf. Its axis in this practical example runs above the femur, in which the axial direction again preferably runs though the system center. In this practical example the epicondyle strips 17rk are mounted fixed on the epicondyle support body 17rf and the epicondyle holders 18f with pads 18g are mounted on the epicondyles by screw guides 18e.

Extremity extension braces can be positioned on the soft tissues of the humerus or femur in the form of belts, plates or shells 17p or directly on the bone in the form of extension screws 17za in the system bore (in which the central bore continues the system bore) or in the form of extension plates 17w-17z, 17za-17zr screwed onto the bones with bone screws 17x on the side facing away from the system bore 13y, in which the extension plates 17w-17z, 17za-17zr have a central hole 17wa in the extension of the system bore 13y.

Extremity extension braces 17p, 17w-17z, 17za-17zr have connection structures or connection devices 17z for the extension arm 17o or a control device 13 mounted on the extension arm or to its tool guide 13a or to a bushing 13b in the tool guide. In order to compensate for deviation of the axis of the central bore 17wa from the axis of the system bore 13y an extremity extension brace can be designed so that it consists of an extension plate 17w, which supports an adjustment and connection body 17zb with the connection device 17z to pivot and be adjusted (see FIG. 33) or the bone screws 17x are supported in a recess 17zj of the extension plate 17w radially and axially as free of play as possible, in which both bone screws 17x are driven with the same rotational speed via a gear mechanism 17zo, 17zp so that during screwing in no deviation in the axis occurs.

FIGS. 31 to 34 show extremity extension braces for application on the humerus 8f or femur 10i with receptacles 17z for the extremity holder, the extension arm 17o and the control device 13 or a tool guide 13a or a bushing 13b in the tool guide 13a or forming a unit with them. FIG. 31 shows a cross section through a shell 17p positioned on the soft tissues. FIG. 32 shows an extension screw 17za with central bore 17wa, which has a bone thread 17y for screwing into the system bore. 20d is a milling cutter shaft, 21e a press-in rod, 21g an ejector rod, which are used through the system bore 13y and the central bore 17wa. FIG. 32a shows an extension screw which forms a unit with the bushing 13b of the tool guide 13a.

FIGS. 33 and 34 show longitudinal sections through extension plates 17w which can be screwed onto the bone with bone screws 17x. 17wa is the central bore in the extension plate 17w, which lies in the extension of the system bore 13y. In both variants the bone screws 17x are supported with a lengthened cylindrical part 17zi in corresponding recesses 17zj of the extension plate 17w. The radial support caused by this stabilizes the position of the extension plate 17w relative to the bone.

The extension plate 17w in FIG. 33 has a bulge 17zc in the form of a spherical section roughly in its center, which serves as pivot bearing for corresponding countersinking of the adjustment and connection body 17zb. In this practical example two adjustment screws 17zd are screwed across the longitudinal axis extension plate 17w into threaded holes 7zk of the extension plate 17w through the adjustment and connection body 17zb. In the longitudinal direction of the extension plate 17w there are two holes for screws 17ze, which are supported with their head in recesses 17zh of the extension plate 17w. Screw sleeves 17ze are screwed onto these screws, which have receptacles 17zf for a rotary tool (for example a slit for a slotted screwdriver).

The bone screws 17x in FIG. 34 are also supported as free of play as possible in both directions axially in addition to radial support in the recess 17zj, for example, by a plate 17zl. The bone screws 17x have gears 17zo above the plate, which cooperate with a gear 17zp in the center. This gear 17zp with its hollow axis 17zq lies on the axis of the system bore and has a hole together with its shaft 17zq which corresponds to the diameter of the system bore 13y. Outside the receptacle structure 17z the hollow shaft 17zq has a structure 17zr for a rotary tool (for example, hexagon, Pozidriv, etc.).

The axis of the tool guide 13a of the control device 13 coincides with the system axis 13x, which runs through the system center 13w and is adjusted to it. Precise control of the system bore 13y and guiding of the milling cutter shafts 19b, 19m, 20d, 20r as well as the press-in/press-on rods 21e, 22m is made possible on this account.

FIG. 35 shows a view of a control device from the foot side of the patient and FIG. 36 shows the same control device from the side. The tool guide 13a is supported to pivot in the pivot bearing 13t on one side and a template holder 13m on the other side. Another template holder 13m extends roughly at right angles to the first one. Both have longitudinal guides 13q in which a support part 13ma of the template is movably mounted. This supports the template 13n in a pivot device 13p. The template contains linear structures 13r and concentric circles 13s. The planes of the two templates are at right angles to each other. The parallel structures 13r are arranged so that a roughly perpendicular projection to the plane of the template 13n images the structures on the system axis (which corresponds to the axis of the tool guide) and the concentric structures on the joint center point.

FIGS. 37 to 40 show practical examples of the tool guide. The tool guide 13a in FIG. 37 is slit and provided with a clamping screw 13c to fasten the bushing 13b in the tool guide 13a. A template holder 13m is fastened on the tool guide 13a, the other template holder 13o on the bushing 13b of the tool guide 13a. The tool guide 13a in FIG. 38 has a clamping device 13b, which is designed as a tightening grip clamping device. The drill for the system bore 13e with a measurement element 13f introduced on it is situated in the bushing 13b of the tool guide. FIG. 39 shows a cross section through a swiveling tool guide 13a. It contains a shaft support or a hinge 13dv, which supports the swiveling part 13du of the tool guide, which is closed with a clamping screw 13c. The tool guide 13a in FIG. 40 is provided with an inside thread, in which a screw bushing 13g and a pressing rod 13h with measurement body 13i are screwed. A measurement strip 13j with a pivot device is mounted on the tool guide 13a to be moved longitudinally and fixed with the screw 13k. It is angled on the measurement end and has a measurement edge 13l there, which checks the press-in depth together with the measurement body 13i.

FIG. 41 shows a transparent view from the side of a control device 13 with tool guide 13a and sliding guide 13va as well as a preferably electronically-controlled drive 13vf of the drive motor 13vb supported on the sliding guide 13va. A screw 13vd, which is supported in a body 13vc connected to the drive motor 13vb with screw guide and in an axial bearing 13ve serves as longitudinal guide and which is driven by a preferably electronically-controlled drive motor 13vf or stepping motor.

FIGS. 42 to 44 show practical examples of tool guides 13a which integrate the function of extension guide 17l, 17m, 17ma, 13da-13do. The function of the pivot bearing 17q of the extremity holder 17r is additionally integrated in FIG. 44. The extension device in FIG. 42 consists of a lever arrangement. The bushing 13b of the tool guide 13a has a ratchet-like snap-in structure 13da on one side, which releasably blocks the movements of the bushing 13b in one direction through a snap-in lever 13db that can be acted upon by a spring 13dc. A lever 13df with a handle 13de has recesses 13dg for pivots. It therefore engages the pivots 13dh of a strip 13di, which is supported on the tool guide 13a (13dh) as well as the pivots 13dh on the bushing 13b and extends them. The bushing 13b has the receptacle structure 13ba for the extremity extension braces 17p, 17w-17z, 17za-17zr on the other end. The extension in FIG. 43 is accomplished in that the tool guide 13a supports a bushing with inside thread 13dj with longitudinal adjustment and can be fastened by the screw 13c. An extension bushing 13dk is screwed into the bushing 13dj. This is supported with an axial bearing 13dl relative to the bushing 13b of the tool guide 13a. The tool support bushing 13b has a connection structure 13ba on the other end for the extremity extension brace 17p, 17w-17z, 17za-17zr. The extension bushing 13dk has a tool receptacle 13do, which can have the external shape of hexagon nut or, for example, a rotor lever or lever with ratchet. By turning out the extension bushing 13dk the extension is carried out. In order to stabilize the tool guide bushing 13b against rotation during the extension process, it is equipped with longitudinal groups 13dm in which correspondingly shaped protrusions 13dn of the bushing with inside thread 13dj engage. A transparent view of a control device 13 is shown in FIG. 44, which integrates the pivot bearing 17q of the extremity holder 17r in addition to the extension function, as well as an adjustment in the device of this pivot bearing. The tool guide 13a with extension device corresponds to the one in FIG. 43 except that here the tool guide 13a simultaneously functions as annular bearing 13dp. An adjustment bushing 13dq is supported on this annular bearing 13dp, which can be locked with a clamping screw 13ds against the annular bearing 13dp. The adjustment bushing 13dq also has a snap-in pin 13dt, which can engage in a hole of the annular bearing 13dr of the extremity holder 17r and in so doing the extremity holder 17r can be snapped into a pre-adjusted pivot angle position.

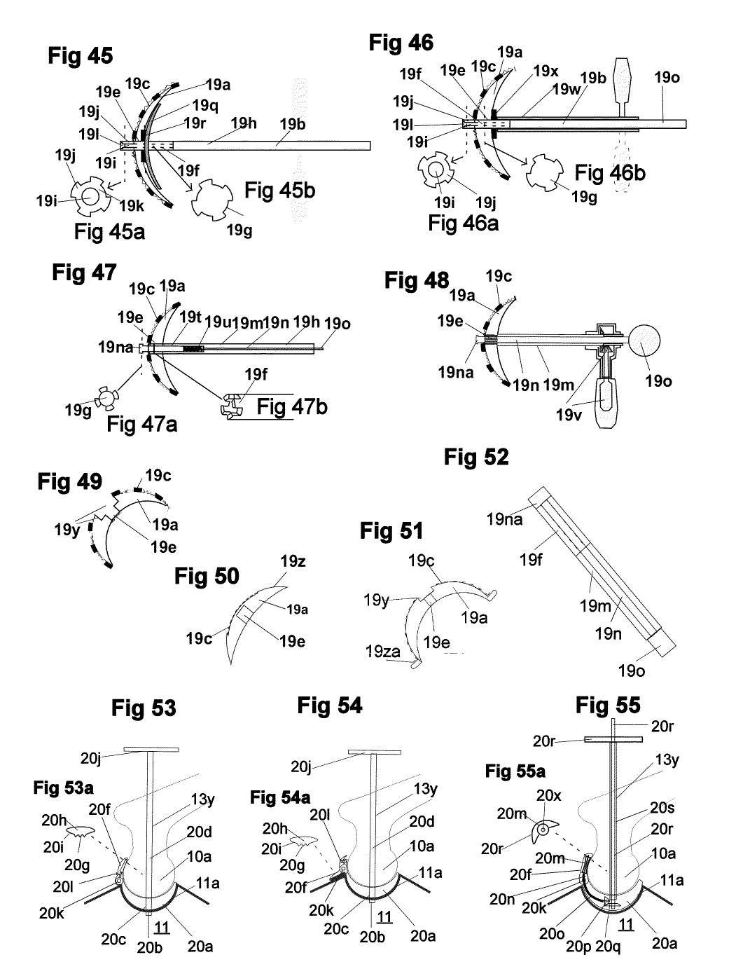

FIGS. 45 to 52 show milling cutters for the narrow joint gap generated by the extension device 17, which are driven and guided through the system bore 13y as well as the tool guide 13a of the control device 13 in the direction of the system axis 13x, which runs through the system center 13w. In order to be introduced into the narrow joint gap, the milling cutter bodies 19a must be concave on the back (i.e., on the side not provided with the milling blades 19c) or be adapted to the shape of the joint head. The cutter shafts 19b, 19m are inserted through the tool guide 13a or its bushing 13b and the system bore 13y into the milling bodies 19a and guided. The milling cutters for the acetabulum preferably have devices with which the milling surfaces protrude forward so that they can be inserted into a lengthened system bore 13z in the joint socket 9b, 11a so that additional support is produced which prevents lateral deviation of the milling cutter 19a.

The milling cutter shaft 19b with its front part 19f profiled to transfer the torque is mounted axially movable in FIG. 45 in a correspondingly profiled central hole 19e of the milling cutter element. An example of such a profile 19g is shown in FIG. 45b. The center and rear (at least the center) part of the milling cutter shaft 19h preferably has a round cross section. Axial force transfer occurs over the joint head or over a shell 19q, which is acted upon by the joint head and is supported relative to the milling element by an axial bearing 19r. The tip of the cutter shaft 19b is tapered on an axial shaft 19i, on which a profiled body 19j is mounted to rotate, whose external profile corresponds to that of the front part 19f of the milling cutter shaft 19b. A cross section 19k through the profiled body 19j is shown in FIG. 45a. A stop washer 19l ensures its axial guiding.

FIG. 46 shows a milling cutter corresponding to FIG. 45 in which the force acts in the axial direction of the milling cutter body 19a through a sleeve 19w on the milling cutter shaft 19b, said sleeve being supported by a bearing 19x relative to the milling cutter body 19a. FIG. 47 shows a corresponding milling cutter having a hollow milling cutter shaft 19m, which drives the milling cutter body 19a both axially and with reference to transfer of torque with a crown 19f (oblique view in FIG. 47b), in which case an internal shaft is supported in the hollow milling cutter shaft 19m. In the front part 19t of the hollow milling cutter shaft 19m this has a greater diameter and is acted upon axially by a spring 19u. For easier passage through the milling cutter body it protrudes on the rear end 19o of the hollow milling cutter shaft 19m. On the tip of the milling cutter internal shaft a profiled punch 19na (see cross section in FIG. 47a), which is provided with a profile 19g, permits passage through the profile 19g of the hole in the milling cutter body. FIG. 48 shows a corresponding practical example in which, instead of a crown on the hollow milling cutter shaft 19m, a multitooth profile 19e produces this drive. In this example the hollow milling cutter shaft 19m is driven via a drive with an angular transmission 19v and the milling cutter internal shaft 19n carries on its back 19o a button to facilitate passage of the profiled punch 19na on the tip of the milling cutter internal shaft through the profiled receptacle 19e of the milling cutter body. FIG. 49 shows a milling cutter body 19a with a central recess 19y in order to accommodate a bone cylinder from milling. The bone cylinder provides the lengthened system bore 13z to guide the milling cutter shaft 19b and the milling cutter internal shaft 19n.

FIG. 50 shows the milling cutter body 19a for the second milling passage, which then serves for milling out of the bone cylinder. The smooth surfaces on the edge 19z serve here as milling depth stop. FIG. 51 shows a milling cutter body that largely corresponds to that in FIG. 49 in which the milling cutter edge, however, is offset and has milling cutter blades on the bottom of the offset edge 19za.

FIG. 52 shows a hollow milling cutter shaft 19m, its milling cutter internal shaft 19n and its profiled punch 19na on the tip of the milling cutter internal shaft 19n corresponding to those in FIG. 47 except that the milling cutter internal shaft 19n has a constant diameter over the entire length of the hollow milling cutter shaft 19m. The rear end 19o of the milling cutter internal shaft 19n has a widened diameter, which, however, is smaller than the diameter of the hollow milling cutter shaft 19m (the milling cutter internal shaft 19n rotates better on this account). Torque transfer to the milling cutter body 19a occurs through a profiled area 19f as in FIG. 45.

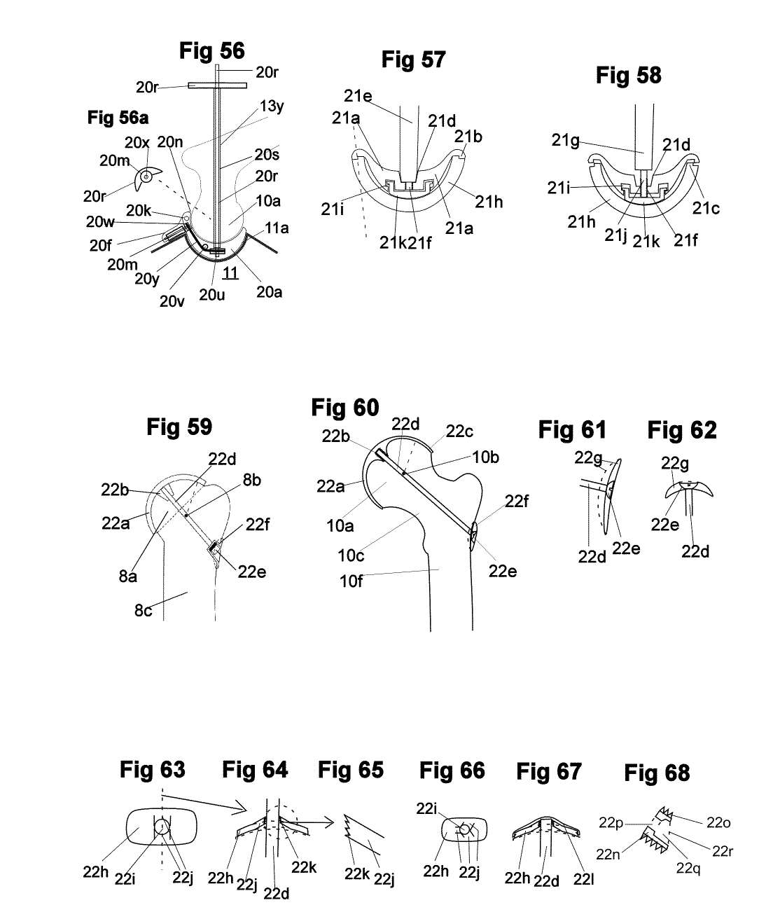

FIGS. 53 to 56 show milling cutters for the narrow joint gap created by the extension device 17, which is driven and guided through the system bore 13y and the tool guide 13a of the control device 13 in the direction of the system axis 13x, which runs through the system center 13w. They are used to ream out osteophytes on the socket edge (FIGS. 54 and 56) or on the edge or transition of the joint head (FIGS. 53 and 55).

FIGS. 53 and 54 show an oscillating manual or machine drive and have milling cutter blades 20g mounted on the milling arm 20f. 10a is the femur head, 11 the pelvis, 11a the acetabulum, 20a is the joint gap shell 20a of the milling cutter, which is inserted into the acetabulum and has a pin 20b, which is inserted in the lengthened system bore 13z. 20f is the milling arm, which has the milling cutter blades 20g on the bottom. FIGS. 53a and 54a show a cross section 20h through this area. The rising smooth surfaces 20i adjacent to the milling surfaces favor sliding on the exostoses. The milling arm is supported in the pivot bearing 20k on the joint gap shell 20a. It has an adjustment or stop screw 20l, with which the milling depth can be sent. The milling arm is preferably spring-loaded (which is not shown). The joint gap shell 20a has a central receptacle 20c for the milling cutter shaft 20d. It can be driven by oscillating movements via a handle 20j or by a machine.

FIGS. 55 and 56 are constructed similar to FIGS. 53 and 54. However, they house a rotating milling cutter 20m in the milling arm 20f. Its milling cutter shaft 20x in the milling device for the femur head is preferably driven via a bevel gear 20p and a flexible shaft 20o. The bevel gear 20p is driven by the bevel gear 20q on the drive shaft 20r. The milling cutter 20m is covered by a cover hood 20r, which protects the surrounding tissue from milling injuries. The cover hood 20r or the milling arm 20f has a stop surface 20n, which adapts the milling to the desired milling contour. For milling of the acetabulum edge and its surroundings the milling cutter shaft drive preferably occurs via a belt pulley 20u on the drive shaft 20r via a drive belt 20y. This is guided around an idler 20v and drives the milling cutter 20m on the milling cutter shaft 20x via a belt pulley 20w. Here again a stop surface 20n for regulation of the milling depth is provided on the milling arm 20f.

Insertion of the prosthesis shells 22a, 21h according to the press-fit method requires press-in or press-on rods 21e, which are applied by the tool guide 13a of the control device 13 and the system bore 13y and are advanced in the direction of the system axis 13x and guided, in which case they pass through the system center 13w. The prostheses 22a, 21h and prosthesis shell holder 21a are shaped so that they can be introduced into the narrow joint gap created by the extension device 17. Pressing in or pressing on preferably occurs through a lever press-on device 22l according to FIG. 69, which has a force display and/or force limitation as well as preferably a measurement device 13ja, 13jb, 13j-13l for measurement of the press-on or press-in path. Separation of the prosthesis shell holder 21a from the pressed-in shell 21h occurs with an ejector rod 21g through the system bore 13y.

FIG. 57 shows a prosthesis shell holder 21a with central recess 21d for the press-in rod 21e. The prosthesis shell holder 21a is shaped so that it has an edge strip 21c opposite the prosthesis 21h with which a releasable snap-in connection to the prosthesis shell 21h is produced.

FIG. 58 shows the ejection process of the prosthesis 21h from the prosthesis shell holder 21a. The ejector rod 21g is screwed into the central bore 21f of the recess 21d with is front part 21j provided with thread. It exerts pressure on the round ejector element 21k, which is supported in an annular guide 21i with axial play—and therefore separates the prosthesis shell 21h from the prosthesis shell holder 21a.

In surface replacement prostheses 22a a preferably rigid press-on rod 22d inserted through the system bore 13y into the central receptacle 22b of the prosthesis is required for an also axially exact press-on process on the joint head 10a, 8a. The inner edge area of a femur head prosthesis shell 22a is preferably shaped as a steep cone 22c. The fixation rod 22d required to achieve reliable primary stability, on the other hand, should preferably have a flexibility adapted to the bone in order to avoid shear forces which would promote loosening. It creates the fixation pressure by counter plates 22f-22l or counter screws 22n in the system bore 13y in the femur shaft or on the outside of the femur shaft 10f.

FIGS. 59 and 60 show surface replacement prostheses 22a on the humerus head 8a or on the femur head 10a. The fixation rod 22d runs through the system bore 13y, the femur head 8a [sic: humerus head] or femur head 10a or through the center point of the joint head 8b, 10b, the femur 8c or femur neck 10c and femur shaft 10f and is screwed onto the counter plate 22f with a screw 22e. The surface replacement prosthesis 22a of the femur head 10a preferably has a conical shape on the inside in the edge area 22c or a surface corresponding to a steep truncated cone.

FIG. 61 shows a longitudinal section and FIG. 62 a cross section through a flat, bulged counter plate 22g with a countersunk screw 22e.

FIGS. 63 to 67 show counter plates designed as shaft clamping disks 22h. The disks 22h are preferably arched. They are adapted to the bone surface on this account and the spring-loaded clamping strips 22j are in a slightly angled position relative to the plane of the central bore 22i. FIG. 63 shows a view of a disk 22h with two spring-loaded clamping strips 22j, FIG. 64 a cross section with the introduced fixation rod 22d, FIG. 65 an enlarged cross section through a spring-loaded clamping strip 22j with details of the sharp edges 22k from FIG. 64. FIG. 66 shows a view of a clamping disk 22h with three spring-loaded clamping strips 22j. FIG. 67 shows a cross section with inserted fixation rod 22d, which is cut off flush with the clamping disk 22h. The clamping disk 22h and fixation rod 22d are covered by a cover cap 22l.

Both the extension plates and counter plates can be equipped for better force introduction onto the bone with an adapting or flexible substrate (for example, silicone) or the counter plate can be mounted with a bone cement substrate. As an alternative to a counter plate a counter screw 22n can be used as anchor for the fixation rod 22d. It is screwed far enough into the system bore 13y, i.e., into the corticalis and/or spongiosa of the bone, so that preferably no overhang exists above the bone surface. The length of the counter screw can correspond as a maximum to the length of the system bore.

FIG. 68 shows the schematic longitudinal section through a counter screw, 22n is the body of the counter screw, 22o the thread adapted to the corticalis or spongiosa, 22p the bore for the fixation rod, 22q the widened recess for the head of the fixation rod or screw and 22r the oblique back side of the counter screw in order to achieve a flat closure with the bone surface.

For axial guiding of the milling cutter shafts 19b, 19m, 20d, 20r and possibly the press-in/press-on rods 21e, 22m the tool guide 13a and system bore 13y are used. Adjustment and control of the advanced path occurs through at least one measurement device 13j on the extension arm, on the control device 13 and on the tool guide 13a of the control device 13 as well as through a fixed or adjustable measurement element 13f, 13h on the milling cutter shafts 19b, 19m possibly on the press-in/press-on rods 21e, 22m. Adjustment of the measurement device 13j or measurement body 13f, 13h to the anatomical circumstances occurs through the measurement probe 24, which is introduced to the joint gap through the system bore 13y.

FIG. 69 shows a transparent view of an extension device with measurement probe 24 for the joint head for adjustment of the measurement device 13j or the measurement body 13f, 13h as well as a measurement device 13j-13l on the tool guide 13a. In this practical example a lever press-on device 21l is mounted on the bushing 13b of the tool guide 13, which has a measurement device 13j-13l corresponding to that on the control device 13. This has the advantage that the lever press-on device 21l can also be used for axial guiding of the milling cutter and checking of the press-on depth of the prosthesis shell can then occur with the adjusted measurement device without requiring repeated measurement by the measurement probe.

FIGS. 70, 71 and 72 show a view from the top, rear and from the side of the head of this probe. FIG. 73 shows an enlargement of the handle with button 24i and adjustable measurement body 24l. The measurement probe 24 consists of a thin, long hollow cylinder 24a, which is introduced through the system bore 13y. The inside diameter is widened in conical-cylindrical fashion on its head (24b), a short stop strip 24c is supported in it whose width corresponds roughly to the diameter of the probe. The stop strip 24c is equipped in the practical example on the side with which it stops against the bone with two cutting blades 24d that serve for grasping of the bone surface in this area. This stop strip 24c is mounted to rotate on the probe on the axial shaft 24e, in which the pivot point lies to the side of the center axis of the probe. The stop strip 24c contains a bore 24f, which also runs to the side of the axis of the probe with the stop strip 24c positioned transverse to the axis of the probe, but on the side opposite the bearing axis. A steel wire 24g is supported in this hole, which passes through the inner hole of the probe to the other end, where it is held in a button 24i which is supported in the probe in a longitudinal guide 24h. This button 24i is acted upon by a spring 24j. The probe in the area of the button 24i has two lateral protrusions 24k, which serve as grips for the index and middle finger (the button is activated with the thumb). The measurement probe 24 or its hollow cylinder 24a is equipped in FIG. 73 with a perforated measurement body 24l, which is mounted on the probe adjustably with a clamping device (for a fixed measurement device). The lever press-on device 21l carries a holder with a pivotable measurement strip 13j, which is adjusted on the measurement body 13f with the measurement edge 13l.

An expansion device 23 is required, if the capsule-ligament apparatus of the joint is very resistant or shrunken by arthrosis. The extension force applicable by the extremity extension brace 17p, 17w-17z via the soft tissues or via the bone screws of the extension plate 17w-17z is limited (about 1-2.5 kN). A much higher extension force is possible with the expansion device 23 (about 10 kN). The capsule-ligament apparatus can be extended far enough that the extremity extension brace can perform the required extension afterward. The extension screw 23a is either provided with a bone thread and is screwed into the system bore 13y in which, for example, the system bore 13y in a first drilling process is applied with a smaller diameter (for example, 5 mm instead of 6 mm of the final system bore), in which a 6 mm extension screw with bone thread 23a is then inserted—or it has a thread with which it is screwed into a threaded hole of a screw shell 23b through the system bore 13y. Outside of the bone the extension screw 23a can carry, for example, a measurement or adjustment device 23h (see FIG. 75), which serves to adjust the extension path. However, the measurement device 13j-13l of the control device 13 is preferably used (see FIG. 74), in which a measurement body 13f is then provided on the extension screw 23a. The extension screw presses in both cases against a pressure shell 23c. This is formed for introduction into the narrow joint gap created by the extension device so that it consists of a roughly 1.0 to 3.0 mm thick circular or oval spherical shell, which is arched roughly according to the bulging of the joint. It can have a preferably curved stem 23e with handle 23f and if required a swivel device as in FIG. 80 or 83. The screw shell 23c and pressure shell 23b are introduced to the joint gap elastically or releasably connected to each other.

FIG. 74 shows a transparent view from the top of a practical example in which the extension screw 23a exerts pressure on the pressure shell 23c as a bone screw in the system bore 13y. Checking of the extension path occurs through a measurement body 13f on the extension screw 23a and the measurement device 13j-13l of the control device 13. The extension screw has a tool receptacle 23j.

To check the extension and adjustment of the optimal bending angle of the extremity the measurement of force development of the extension can be favorable. FIG. 74a shows a longitudinal section through a pressure shell 23c with pressure sensors 23p and FIG. 74b shows a longitudinal section through the front part of an extension screw 23a with a pressure sensor 23k. A pressure receiving shell 23o is supported in the pressure shell 23c via pressure sensors 23p and a punch 23l, which acts on the pressure sensor 23k in a recess 23m serving as a longitudinal guide in the tip of the extension screw 23a. The measured values and/or power supply occur via cable in cable channels 23n.

FIG. 75 shows a longitudinal section through a practical example of an extension device with screw shell and screw wing 23g on the extension screw 23a. The pressure shell 23c in this practical example is fastened on the stem 23e with a spring steel sheet 23d. It is held on the screw shell 23b by the spring steel sheet 23d. The extension screw 23 is screwed into the central threaded hole 23i of the screw shell 23b. It carries on its front end a thread. The extension screw in this practical example has its own measurement device 23h.

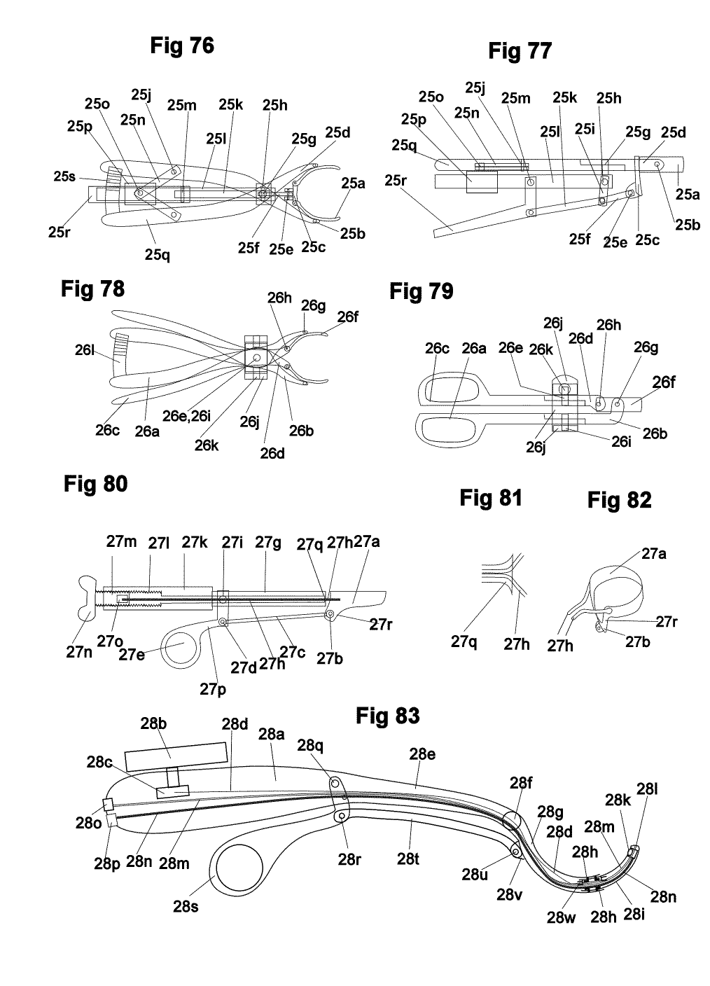

FIGS. 76 to 82 show pivot-gripping tools 25-27, which are equipped with a pivot function or swivel function in addition to the gripping function of a gripper, which 1. is required because of the angle difference between the soft tissue channel leading to the joint and the angle of the joint gap created by the extension device and 2. in order to co-pivot the milling body 19a and prosthesis shells 21h, 22a for pivoting into the joint gap in a combined forward and pivot movement, which requires adjustment with the pivoting back movement of the joint head through the extension device 17. This achieves a situation in which they are pivoted in so that the milling cutter body 19a or prosthesis shells 21h, 22a or prosthesis shell holder 21a are brought into a position in which the milling cutter shafts 19b, 19m, 20d, 20r and press-in/press-on rods 21e, 22m can be inserted into them through the system bore 13y. The pivot gripping tools 52-58 according to the invention can consist of a gripper 25q, 25d whose jaws 25d are supported on the gripping strips 25a, in which an additional handle (for example, toggle lever) 25r pivots a swivel strip 25c via a swivel rod 25f, 25k on which the gripping strips 25a are supported or it consists of two grippers 26a-26b, 26c-26d which are oppositely supported in at least one pivot device 26k in which the pivot axis runs roughly at right angles to the axial direction of the gripper shafts 26i, 26e and the jaws 26b, 26d of the two grippers are supported on different sites of the gripping strips 26f so that the movement of handles 26a, 26c toward each other produces pivoting of the gripping strips 26f, or it consists of a handle 27k with tightening device 27m-27o and a tightening band 27a as well as an additional handle 27p, which pivots the tightening band 27a via a swivel rod 27c in which the tightening band 27a is tightened via wires 27h or strands or bands by the tightening device 27m-27o.

FIG. 76 shows a front view and FIG. 77 a longitudinal section through a practical example of a pivot-gripping tool 25, which carries out the pivot process through a swivel strip 25c and swivel rod 25f, 25k. 25a are the two gripping strips, each of which has a spherical head 25b with stem, with which they are supported on the gripping jaws 25d. The gripping strips 25a are oppositely supported on the swivel strip 25c. The swivel strip 25c has on the lower end a protrusion with a support 25e for a divided swivel rod 25f and 25k. This swivel rod 25f, 25k is supported in the division location in a pivot arm 25i, which is mounted on the main strip 25l and on the other end the swivel rod is mounted on the toggle lever 25r, which is again supported on the main strip 25l in the joint 25m. The main strip 25l is mounted on the fastening 25h of the shaft of the gripper joint 25g and is additionally supported in a linear guide (a bushing 25p) between the gripping arms 25q. The bushing 25p has a pivot 25o on which two strips 25n are supported, which are supported on the other end 25j on the gripping arms 25q. One of the gripping arms 25q has a blocking device 25s, which blocks or fastens the gripping arms 25q.

FIG. 78 shows a transparent view and FIG. 79 a longitudinal section through a practical example of a pivot-gripping tool 26, which consists of two grippers. The gripper that consists of the gripping arms 26a and gripping jaws 26b closes the jaws 26b during compression of the gripping arms 26a (in this example with gripping eyes). The grippers with the gripping arms 26c and jaws 26d are supported in their joint 26e so that compression of the gripping arms 26c causes opening of the gripping jaws 26d. 26f are the gripping strips, which have supports 26g and 26h for the gripping jaws 26b and 26d. The closing jaws 26b are supported in the joint 26i, the opening jaws in the joint 26e. The lower gripper 26a carries a box 26j on the gripping shaft 26i, which runs upward to the side of both grippers and has a transversely running shaft 26k in the upper part on which the shaft 26e of the upper opening gripper is mounted. The lower gripper carries a blocking device 26l. The upper one, which, however, is not shown for reasons of clarity, preferably does too. After closure of both gripping arms 26a, 26c, the milling cutters 19a, 20a and prosthesis shells 21h, 22a are fixed. If the two grippers are now moved toward or away from each other the milling cutters 19a, 20a or prosthesis shells 21h, 22a are pivoted.

FIG. 80 shows a practical example of a pivot-gripping tool 27 with tightening device. 27a shows the continuous tightening band, which is curved downward to the side of the main strip 27g. There it carries the support 27b for the swivel rod 27c. This is mounted on the toggle lever 27p and bearing 27d. The toggle lever is again supported on the main strip 27g in the bearing 27i. It has a large eye 27e as a handle for passage of a finger. The main strip 27g is hollow. The wire cables 27h that close the tightening band 27a run in it (see also FIGS. 81 and 82). A threaded hole 27l in which a tightening screw 27m is supported is situated in the handle 27k. This supports the ends of the wire cable 27h in a body with axial bearing 27o. The tightening screw 27m is operated by the wing 27n. FIG. 81 shows the opening of the main strip 27g from FIG. 80 in a view from the top. It is widened funnel-like 27q both in the horizontal and vertical direction. The wire cables 27h of the tightening band 27a run in this opening. FIG. 82 shows the continuous flexible tightening band from FIG. 80 in an oblique view, which consists here of a spring steel sheet. The tightening band 27a forms a continuous ring which is arched downward U-shaped on the side against which it contacts the tightening wires 27h (27r). At this location the ring has a protrusion with a support 27b for the swivel rod 27c. In the upper area two tightening wires (strands) 27h are attached at the sites lying next to the upper edge.

Before implantation of the prostheses inspection of the milling surfaces is required. For the joint socket this occurs through an endoscopy instrument which is inserted through the system bore 13y. To check the joint head an inspection device 28 is used, which can be introduced into the narrow joint gap. It consists of a handle 28a with stem 28e and preferably a pivot bearing 28f which permits pivoting of strips 28g, 28i. For this purpose it is equipped with a pivot device. The joint gap strip 28g consists either of a front and rear strip so that the strip 28i supported in it can be partially pivoted between them or consists of a closed body which tapers to a tip toward the support and in so doing permits pivoting around a large angle. Pivoting occurs in this practical example in that the strand 28d on the other end is fastened on a coil 28w fixed on the supported strip so that rotation of the rotary button 28b on the handle 28a moves both strands on the coil 28c and in so doing causes pivoting of the support strip 28i. Pivoting is also possible, for example, through a bevel gear on the joint strip support 28h, which is driven by a small bevel gear and a micromotor or flexible shaft. The supported strip 28i contains a minicamera 28k and the nozzle 28l or an opening for the rinsing liquid. Both strips are shaped according to the joint gap.

FIG. 83 shows a transparent view of a practical example of an inspection device of the joint gap for the hip joint. A connection 28o for the rinsing tube 28m and the cable 28n of the video camera are situated in the handle 28a. On the transition to the stem a pivot bearing 28q is found for the toggle lever 28s, which has a handle with eye. In the knee area it has a support 28r for a swivel rod 28t which is mounted in a bearing 28u on a protrusion 28v of the joint strip 28g. The joint strip 28g is mounted opposite the stem in the pivot bearing 28f. In this example it has two supports 28h, one of which on the inside and the other on the outside support the front and rear shells of the supported strip 28i. Here again a bearing shaft with a hole or recess for the wires 28n of the minicamera 28k and the rinsing tube 28m is possible.

1. Apparatus for performing a hip or shoulder joint endoprosthesis implantation with
an operating table (1),
an extension device (17, 18) for extension and pivoting of a joint head (8a, 10a) from a joint socket (9b, 11a) of a patient and
a fixation device (14-16) for fixation of a body of the patient in the area of a hip or shoulder joint to produce a controlled position relation of the joint socket relative to the joint head (8a, 10a) to be extended with the extension device (17, 18), in which the fixation device (14-16) has adjustable holding devices for fixation of a pelvis or alternatively a shoulder,
in which the extension device (17) has an extension guide (17l, 17m, 17ma, 13dj, 13da-13do), whose axis runs parallel to a system axis (13x), as well as an extension arm (17o), an extremity extension brace (17p, 17w-17z, 17za-17zr) for the femur or alternatively for the humerus, an extremity holder (17r) adjustable relative to the extremity extension brace (17p, 17w-17z, 17za-17zr) for the leg or alternatively for the arm,
in which the extension device (17) is provided with at least two system axis-pivot devices (17b-17l, 7n), in which one axis of at least one system axis pivot device (17b-17l) runs through a system center (13w) and the system axis pivot devices (17b-17l, 7n) are arranged so that system axis (13x) of the extension device (17) is adjustable relative to the system center adjustment devices (2-2d, 17b-17c, 17l, 7n) and fixation devices (14-16, 7) to a planned inclination and anteversion angle (5), in which the longitudinal axis of the extension guide (17l, 17m, 17ma, 13dj, 13da-13do) runs parallel to the system axis (13x),
in which the extension arm (17o) of the extension device (17, 18) is connected to the holding devices of the fixation device (14-16) via the system center-adjustment devices (2-2d, 17b-17c, 17l, 7n) in three spatial axes, which are spatially arranged so that by relative displacement of the extension device relative to the fixation device the system center (13w) of the extension device (17-18) can be adjusted to the joint center point (8b, 10b) of the hip or shoulder joint established by the fixation device (14-16)
in which on at least one of the following elements connection devices for connection to the operating table are provided:
system axis pivot devices (17b-17l, 17n),
system center adjustment devices (2-2d, 17b-17c, 17l, 7n),
fixation device (14-16, 7),
extension device (17, 18),
and in which a control device (13) is provided, which has a tool guide (13a) adjustable to the system axis (13x), which provides alignment of a drill (13e) for a system bore (13y) and milling and prosthesis implantation tools on the system axis (13w).

2. Apparatus according to claim 1, in which the fixation device for the pelvis (14-16) includes at least four of the following adjustable holding devices adapted to the bone shapes:
at least one rear pelvic brace (14h, 14i, 14zi, 14r, 14x, 15c, 15ia),
at least one ischial tuberosity support (14h),
at least one anterior iliac spine support (14i, 14r),
one at least one-part front pelvic brace (16),
at least one anterior superior iliac spine holder (14q, 14w),
at least one lateral pelvic support (14m, 14x, 14xa, 14zl),
an ischium-pubic bone support (15),
a symphysis support (15n-15s, 15v),
at least one iliac crest holder (14p, 14s).

3. Apparatus according to claim 1, in which the fixation device for the shoulder can be mounted on the base (7) or operating table (1) and the fixation device includes at least three of the following adjustable holding devices adapted to the bone shapes:
adjustable belts over at least one shoulder and the thorax (7a, 7b),
at least one clavicle support with pad strips (7k),
at least one medial shoulder blade brace (7l),
at least one lateral shoulder blade brace (7m),
at least one holder of the angulus inferior scapulae (7d, 7r),
at least one acromion holder (7h, 7ha),
at least one coracoid process holder (7i, 7k, 7r, 7p, 7q, 7u),
at least one spina scapulae holder (7e),
a spina scapulae fixation screw (7f) with its brace (7g).

4. Apparatus according to claim 1, in which a holding device (17n) with a mounting structure or snap-in structure (17na) for snapping in of a corresponding holder of an x-ray device is situated on one of the devices comprising
the system axis pivot devices (17b-17l, 7n),
the system center adjustment devices (2-2d, 17b-17c, 17l, 7n),
the extension devices (17, 18).

5. Apparatus according to claim 1, in which the extremity holder (17r) includes at least one of the following connections to the extension arm (17o):
a fixed connection in which it forms a unit with the extension arm (17o),
a mounting or snap-in connection (17m),
a support in at least one pivot device (17q),
a support in at least one longitudinal guide (17rb, 17rg, 17rh, 17rm, 18e), and that it includes at least one of the following elements for adjustable support of the extremities:
at least one pivot bearing or arc guide (17q, 17ra, 17rc, 17rd, 17rf-17rh, 17rj, 17rm, 18, 18a-18c),
at least one longitudinal guide as adjustment device (17rb, 17rg, 17rh, 17rm, 18e),
at least one extremity support shell (17s),
at least one adjustable brace (17rh-17rl, 18e-18g) for the epicondyles of the femur (10g) or the humerus (8d).

6. Apparatus according to claim 1, in which the control device (13) has a guide for tool drives (13vb) comprising a longitudinal guide (13va-13vf) or a sliding guide, in which one axis of the longitudinal guide (13va) runs parallel to an axis of the tool guide (13a).

7. Apparatus according to claim 1, in which at least two of the following devices have an electronically-controlled drive for program-controlled coordinated movement:
at least one system center adjustment device (2, 2a-2c, 17b-17c, 17l, 7n),
at least one system axis pivot device (17b-17l, 7n),
the extension guide (17l, 17m, 17ma, 13dj, 13da-13do),
the guide (13va-13vf) for tool drives on the control device (13).

8. Apparatus according to claim 1, in which the control device (13) with the tool guide (13a) is also suitable for assuming functions of the extension arm (17o) in which the control device (13) is mounted on the extension arm (17o) with at least one of the following devices:
a fixed connection so that a unit is present with the extension arm (17o),
at least one mounting or snap-in device (13u, 13ua, 17oa),
at least one adjustment device (13v),
at least one pivot bearing (13t),
and in which the control device (13) with the tool guide (13a) forms at least one of the following devices of the extension arm (17o):
the extremity extension brace (17p, 17w-z),
the extremity holder (17r),
the extension guide (17l, 17m, 17ma, 13dj, 13da-13do).

9. Apparatus according to claim 1, in which the control device (13) mountable on the extension arm (17o) has the tool guide (13a) or a bushing or a hollow element (13b, 13g) in the tool guide (13a), which is combined or can be connected with an extremity extension brace screwed on the humerus or femur bone of the patient in the form of an extension plate (17w-17z) and extension screw (17za).

10. Apparatus according to claim 1, in which the tool guide (13a) is supported in at least one snap-in pivot bearing (13t) on the control device (13).

11. Apparatus according to claim 1, in which the control device has a bearing running in the system axis which adjustably supports coaxially at least two of the following elements:
a pivot device (17q) of the extremity holder (17),
the extension device (17) in the form of an axial longitudinal guide,
the tool guide (13a).

12. Apparatus according to claim 1, in which the extremity extension brace (17p, 17w-17z, 17za-17zr) designed as an extension plate (17w, 17wa, 17z, 17za-17zr) or extension screw (17za) has a connection (17z) to the tool guide (13a) and the extension plate (17w) is provided with screw holes for bone screws (17x) and is combined with an adjustment and connection element (17zb) to the tool guide (13a, 13b) and the extension plate (17w) with the adjustment and connection element (17zb) are connected to each other via an adjustable pivot device (17zc, 17zc-17zh, 17zk) in at least one pivot plane.

13. Apparatus according to claim 1, in which the extremity extension brace designed as extension plate (17w, 17wa, 17z, 17za-17zr) has a connection to the tool guide (13a), and the extension plate (17w) has a bone plate with screw holes for bone screws (17x) and a connection device (17z) to the control device (13, 13a, 13b), in which the bone screws (17x) have a cylindrical part (17zi) outside a bone thread, with which they are supported radially and axially in a corresponding recess (17zj) in the extension plate (17w) and each bone screw (17x) in this area is provided with a gear (17zo), in which the gear (17zo) of the bone screws (17x) is coupled via a coupling gear (17zp) positioned in between and this coupling gear (17zp) has a drive shaft (17zq) and a drive structure (17zr) for a rotating tool.

14. Apparatus according to claim 1, in which at least two of the following devices have an electronically controlled drive for program-controlled coordinated movement:
at least one system center adjustment device (2, 2a-2c, 17b-17c, 17l, 7n),
at least one system axis pivot device (17b-17l, 7n),
the extension guide (17l, 17m, 17ma, 13dj, 13da-13do),
a guide (13va-13vf) for tool drives on the control device (13).

15. Milling cutter for use with an apparatus according to claim 1 with
a milling cutter body (19a) for a narrow joint gap, which forms by the extension device (17) and
a milling cutter shaft (19b, 19m), which can be driven and guided by the control device (13) and the system bore (13y),
in which the milling cutter body (19a) satisfies the stipulation of the narrow joint gap in that it is adapted on both sides at least partially to the shape of a pending joint part and
in which the milling cutter shaft (19b, 19m), which can be inserted in a central receptacle (19e) of the milling cutting body, is shaped according to the control device (13) and system bore (13y), in which a measurement element (13f) is provided on the milling cutter shaft (19b, 19m) as a reference for a measurement device (13j-13l) on the control device (13).

16. Milling cutter according to claim 15 for milling of the joint socket in which the central receptacle (19e) of the milling cutter body (19a) for the milling cutter shaft (19b, 19m) represents a profiled hole (19e) which also only partially passes through the milling cutter body, for axial force exposure through the milling cutter shaft (19b, 19m), in which at least in one front part of the milling cutter shaft (19b, 19m) an internal shaft (19n) is supported to rotate as a guide pin, which has a shape and support that provides for penetration of the milling cutter body and protrusion forward beyond a surface of the milling cutter blade.

17. Milling cutter according to claim 15 for milling of the joint socket in which the central receptacle (19e) of the milling cutter body (19a) for the milling cutter shaft (19b, 19m) is a hole profiled in cross section for a coaxially longitudinally movable support of the milling cutter shaft (19b, 19m), in which the milling cutter shaft is profiled in a front part corresponding to the central receptacle (19e) of the milling cutter body (19a) and the milling cutter shaft has a body (19k) on its front part (19i) reduced in diameter corresponding to its profile, rotatable at the tip and protruding forward beyond the milling cutter body.

18. Milling cutter according to claim 15, in which a first milling cutter body (19a) with a central recess (19y) to the side of the milling cutter blade is provided, said recess sparing a bone cylinder from milling and that a second milling cutter body (19a) is provided, which has central milling cutter blades (19c) to treat the remaining bone cylinder and peripherally has an edge area with a smooth surface (19y) whose bulge continues the bulge of the tip of the milling cutter blade.

19. Milling cutter according to claim 15, in which a milling cutter to ream out osteophytes on the joint socket or alternatively the joint head and for improvement of an offset of a joint head transition has a shell (20a) without milling cutter blades as milling cutter body, on whose edge an adjustable arm (20f) with milling cutter blade (20g) is mounted.

20. Milling cutter according to claim 15, in which a milling cutter to ream out osteophytes on the joint socket or alternatively the joint head and for improvement of an offset of a joint head transition has a joint gap shell (20a) without milling cutter blades as milling cutter body, on whose edge an adjustable arm (20f) with a milling cutter (20m) mounted to rotate in it is pivotable, in which a milling cutter drive occurs by a drive system (20o-20q, 20u-20w) that transfers rotations and drives the drive shaft (20r) of this drive system (20o-20q, 20u-20w) supported in a torsion shaft (20s), in which the torsion shaft (20s) is supported in the system bore (13y) and in the tool guide (13a) and carries out rotation of the shell (20a).

21. Prosthesis shell insertion tool (21) for the socket prosthesis shell (21h) for use in conjunction with an apparatus according to claim 1, in which it includes
a prosthesis shell holder (21a) for a narrow joint cap, which forms by the extension device (17),
a pressing rod (21e) and an ejector rod (21g), both of which are guided and can be driven by the control device (13) and the system bore (13y),
in which the prosthesis shell holder (21a) fulfills the stipulation of the narrow joint gap in that it has at least partially a shape adapted to a milled out joint head on a side facing away from the joint socket and the prosthesis shell holder (21a) centrally has a recess (21d) for a press-on rod (21e), in which in a center of the recess a remaining part of a depth of the prosthesis shell holder is provided with a central hole (21f) for the ejector rod (21g), which has an inside thread and the prosthesis shell holder (21a) is releasably connected to the socket prosthesis shell (21h) and
a central part of the prosthesis shell holder (21a) supports an ejector element (21k) in a guide (21i) on a side facing the socket prosthesis shell, which has a spherical bulge on the side of the socket prosthesis shell which corresponds to that on the inside of the socket prosthesis shell, in which the press-on rod (21e) as well as the ejector rod (21g) are shaped according to the control device (13) and the system bore (13y),
and in which the ejector rod (21g) is provided with a thread on a tip (21j), which cooperates with the thread provided in a center of the recess of the prosthesis shell holder (21f).

22. Prosthesis for a joint head, for use in conjunction with an apparatus according to claim 1, in which a joint gap, which is produced by the system axis pivot device (17b-17l, 7n) and the extension device (17), is so narrowly limited by the anatomical conditions that a socket prosthesis shell (22a) after introduction fills it up and its application onto the joint head therefore can only occur through the system bore (13y), in which an exact press-on direction is stipulated by the system bore (13y), in which the prosthesis includes the following components:
a press-on rod which is shaped rigid and according to the system bore (13y),
a fixation rod (22d) which is shaped for fixation of the socket prosthesis shell (22a) through the system bore (13y) and which is provided with a counter plate (22e-22l) or counter screw (22m),
a socket prosthesis shell (22a), which centrally has a common receptacle (22b) for the press-on rod and the fixation rod (22d).

23. Inspection tool for a joint gap for use in conjunction with an apparatus according to claim 1 for the joint gap produced by the extension device (17), in which it has an arc-like gap strip (28g), on which a similarly shaped hollow strip (28i) is mounted to pivot around an axis (28h), in which the hollow strip (28i) contains a mini video camera (28k) as well as a tube (28m) with rinsing nozzle (28l) for free rinsing and improvement of sight, if required.

24. Gripping tool for use in conjunction with an apparatus according to claim 1 for insertion and removal of milling cutter bodies (19a, 20a), prosthesis shells (22a, 21h) and a prosthesis shell holder (21a) in a joint gap produced by the extension device (17), in which it is formed as a pivot-gripping tool (25, 26, 27), in which it has gripping strips (25a, 26f) or a tightening band (27a) for holding the milling cutter bodies (19a, 20a), the prosthesis shells (22a, 21h) and the prosthesis shell holder (21a) and also has at least one pivot device (25b, 26k, 26g, 26h, 27q) in addition to at least one gripper joint (25d, 26e, 26i) or in addition to a tightening device of a tightening band (27a), whose axis runs roughly at right angles to the axis of the gripper joint (25d, 26e, 26i) in which this pivot device (25b, 26k, 26g, 26h, 27q) carries the milling cutter bodies (19a, 20a), prosthesis shells (22a, 21h) or prosthesis shell holder (21a) held by the pivot gripping tool during pivoting back of the extended joint head by the system axis pivot device (17b-17l, 7n) of the extension device (17).

25. Measurement probe for use in conjunction with an apparatus according to claim 1 for establishing a milling cutter guide relative to a milling depth on the joint head, in which the measurement probe is designed for use through the system bore (13y) and the tool guide (13a) and has a tube (24a) for the system bore (13y), which has a stop strip (24c) that can be swiveled out transversely on the tip (24b) provided for the joint gap generated by the extension device (17), in which this stop strip (24c) is swiveled in and out transversely by a shaft (24d) in the tube (24a) and a handle (24i) on the other end and in which the probe (24a) is equipped with a perforated measurement element (24l) or a marking as reference for the measurement device (13j-13l) on the control device (13), which is mounted fixed or longitudinally adjustable on it.

26. Apparatus for extension of a joint capsule for use in conjunction with an apparatus according to claim 1, in which an extension device (23) has an extension screw (23a) for the system bore (13y) and has a pressure shell (23c) to be introduced in a joint gap, in which the joint gap is generated by the extension device (17).
